SEQUENCE LISTING (9 sequences)

SEQ ID NO: 1 - DNA, 31 bp, synthetic construct
  source 1..31: mol_type = other DNA
tttactgtgagaaaattagagtgttcaaagc

SEQ ID NO: 2 - DNA, 31 bp, synthetic construct
  source 1..31: mol_type = other DNA
tttactgtgagaaaattagagtgtttaaagc

SEQ ID NO: 3 - DNA, 20 bp, synthetic construct
  source 1..20: mol_type = other DNA
tgctttcgcacttgttcatc

SEQ ID NO: 4 - DNA, 20 bp, synthetic construct
  source 1..20: mol_type = other DNA
tgctttcgcactagttcatc

SEQ ID NO: 5 - DNA, 20 bp, synthetic construct
  source 1..20: mol_type = other DNA
tgctttcgcacctgttcatc

SEQ ID NO: 6 - DNA, 22 bp, synthetic construct
  source 1..22: mol_type = other DNA
cagttgggggtattcatattca

SEQ ID NO: 7 - DNA, 20 bp, synthetic construct
  source 1..20: mol_type = other DNA
ggacgtatcatgctggccaa

SEQ ID NO: 8 - DNA, 19 bp, synthetic construct
  source 1..19: mol_type = other DNA
ggaaatagcctccggctca

SEQ ID NO: 9 - DNA, 26 bp, synthetic construct
  source 1..26: mol_type = other DNA
tccttcgtgatatcggacgttggctg

(19) United States
(12) Patent Application Publication
(10) Pub. No.: US 2026/0201483 A1
(43) Pub. Date: Jul. 16, 2026

(54) Blastocystis sp. as a Predictive Biomarker of High Productive Longevity in Dairy Heifers

(71) Applicants: UNIVERSITE DE LILLE, Lille (FR); INSTITUT NATIONAL DE LA SANTÉ ET DE LA RECHERCHE MÉDICALE, Paris (FR); CENTRE NATIONAL DE LA RECHERCHE SCIENTIFIQUE, Paris (FR); CENTRE HOSPITALIER UNIVERSITAIRE DE LILLE, Lille (FR); GD BIOTECH, Douai (FR); INSTITUT PASTEUR DE LILLE, Lille (FR)

(72) Inventors: Magali CHABE, Lille (FR); Eric VISCOGLIOSI, Marquette lez Lille (FR); Nausicaa GANTOIS, Wattignies (FR); Christophe AUDEBERT, Lille (FR); Gael EVEN, Faches Thumesnil (FR); Sophie MERLIN, Neuville en Ferrain (FR)

(73) Assignees: UNIVERSITE DE LILLE, Lille (FR); INSTITUT NATIONAL DE LA SANTÉ ET DE LA RECHERCHE MÉDICALE, Paris (FR); CENTRE NATIONAL DE LA RECHERCHE SCIENTIFIQUE, Paris (FR); CENTRE HOSPITALIER UNIVERSITAIRE DE LILLE, Lille (FR); GD BIOTECH, Douai (FR); INSTITUT PASTEUR DE LILLE, Lille (FR)

(21) Appl. No.: 19/136,438

(22) PCT Filed: Dec. 7, 2023
(86) PCT No.: PCT/EP2023/084652

§ 371 Date: Jun. 6, 2025

(30) Foreign Application Priority Data
Dec. 7, 2022 (EP) 22212049.5

(51) Int. Cl. C12Q 1/6893; C12Q 1/686

(52) U.S. Cl. CPC C12Q 1/6893; C12Q 1/686

(57) ABSTRACT

The invention provides a biomarker, Blastocystis sp., the presence of which, in a fecal sample of a dairy cow at the heifer stage, is indicative of a future high productive longevity, of a richer and more diverse gastrointestinal microbiota, as well as an improved carbon footprint compared to a Blastocystis-negative dairy cow. The invention also concerns methods, including a qPCR method, and kits for detecting Blastocystis sp. in fecal samples obtained from heifers, for predicting productive longevity in heifers, and for improving dairy herd management.

### RELATED APPLICATION

The present application claims priority to European Patent Application No. EP 22 212 049 filed on Dec. 7, 2022, which is incorporated herein by reference in its entirety.

### BACKGROUND OF THE INVENTION

Dairy replacements are the foundation of any dairy enterprise. Improvement of a herd is possible when culled cows are replaced by well-fed, healthy, genetically superior, more efficient, and properly managed heifers.

Dairy herd renewal refers to the number of heifers bred to enter the dairy herd per season. In the short term, it is one of the most expensive items in a dairy unit, but in the medium or long term, it has an impact on milk production of future campaigns and allows for genetic improvement of the herd. In most configurations, the herd renewal is defined by the number of culled cows: i.e., the animals that must be removed from the herd (up to 25%) for economic or sanitary reasons (infertility, mammary infections, etc.) as well as the animals that died during the campaign (5%). A safety margin of 5% is generally added to this to anticipate unforeseen events. Finally, when the dynamics of the herd is stable, the objective is not to exceed a renewal rate of 35%.

Few means are available to make an informed selection of heifers to include in the renewal plan. Genotyping and indexing allow to identify the heifers with the best genetic potential. Genomic indexes can be used to guide the breeders' choice in order to achieve genetic progress more rapidly according to key parameters for the optimization of their herds (milk production criteria, protein or fat content, functional criteria such as resistance to certain diseases or morphology criteria). The heifers with the best genetic potential can for example be inseminated with sexed semen in order to only produce female calves, i.e., future heifers that can be then integrated into the herd. This protocol allows to optimize the genetic progress. The heifers with an intermediate genetic potential can be inseminated with conventional semen, while those with the least potential can be inseminated with beef cattle (industrial crossbreeding in order to produce calves that have higher sale value).

If the objective of breeding is to sustain the production level by reducing costs while having a lower impact on the environment, the criterion of productive longevity can be a decisive element. Indeed, dairy cow longevity is an essential economic trait that can complement the breeding value of productive traits, which is related to the herd duration and the lifetime milk yield of dairy cows. In addition, identification of animals with the potential to fulfill a longer career in dairy production is a major factor for a more sober and more efficient breeding in terms of greenhouse gas emissions and for reaching an optimal size of the milking herd. However, productive longevity is a relatively difficult trait to select for breeding dairy cows due to the low heritability and the many factors influencing longevity in dairy cows. In absence of suitable means, this trait is most often not considered.

Therefore, there is a need in the art for methods and tools for determining productive longevity in dairy cows, in particular for discriminating heifers on their potential to achieve a longer productive life, i.e., to allow an early prediction of their future potential in dairy production. The present invention addresses these needs.

### SUMMARY OF THE INVENTION

By testing 200 heifers belonging to 19 different herds and following them throughout their milk production careers for more than 3 years, the present Inventors have surprisingly observed that Blastocystis sp. colonization in a dairy heifer is associated with a future high productive longevity. On average, dairy cows having completed their lactation career and identified, at the heifer stage, as being infected with the protozoan Blastocystis sp. were found to have a productive longevity 138 days longer compared to non-infected dairy cows (which corresponds to an improvement of around 30% in productive longevity). The increase in productive lifespan occurred without any incidence on yield production and on the intrinsic qualities of the milk produced. The present Inventors have calculated that Blastocystis-negative dairy heifers have a 17.5% higher carbon contribution per kg of milk produced compared to Blastocystis-positive animals, suggesting that a greater productive longevity constitutes a good option not only for improving profitability but also for mitigating Greenhouse Gas (GHG) emissions. The present Inventors have further shown that Blastocystis sp. colonization in a dairy heifer is associated with a significantly greater gastrointestinal microbiota richness and diversity than observed in non-infected animals. Furthermore, the presence of Blastocystis was found to be associated with a drastic decrease in the constitutive genera of the Enterobacteriaceae family, some of which are pathogens responsible for subclinical or clinical bovine mastitis.

Thus, in one aspect, the present invention provides a method for predicting productive longevity in a dairy cow, the method comprising a step of: detecting the presence or absence of Blastocystis sp. in a fecal sample obtained from the dairy cow when the dairy cow is a heifer, wherein the presence of Blastocystis sp. in the fecal sample is indicative of a higher productive longevity in the dairy cow, and the absence of Blastocystis sp. in the fecal sample is indicative of lower productive longevity in the dairy cow.

The present invention also provides a method for predicting carbon footprint of a dairy cow, the method comprising a step of: detecting the presence or absence of Blastocystis sp. in a fecal sample obtained from the dairy cow when the dairy cow is a heifer, wherein the presence of Blastocystis sp. in the fecal sample is indicative of a lower carbon footprint for the dairy cow, and the absence of Blastocystis sp. in the fecal sample is indicative of higher carbon footprint for the dairy cow.

The present invention further provides a method for predicting microbiota «-diversity in a dairy cow, the method comprising a step of: detecting the presence or absence of Blastocystis sp. in a fecal sample obtained from the dairy cow when the dairy cow is a heifer, wherein the presence of Blastocystis sp. in the fecal sample is indicative of a higher microbiota α-diversity in the dairy cow, and the absence of Blastocystis sp. in the fecal sample is indicative of lower microbiota «-diversity in the dairy cow.

In certain embodiments, the methods of the invention are used for testing a heifer belonging to a dairy cattle breed, in particular to a dairy cattle breed selected from the group consisting of Holstein-Friesian, Montbéliarde, Normande, Jersey, Brown Swiss, Guernsey, Ayrshire, and Milking Shorthorn.

In certain embodiments, the heifer is 6 months old or older and is 36 months old or younger, preferably the heifer is between 6 and 20 months old, or between 6 and 18 months old.

In certain embodiments, the step of detecting the presence or absence of Blastocystis sp. in the fecal sample is carried out using a method selected from the group consisting of microscopic examination, in vitro culture, molecular detection, and any combination thereof.

In certain embodiments, the step of detecting the presence or absence of Blastocystis sp. in the fecal sample by molecular detection is carried out using a molecular detection method selected from the group consisting of immunoblots (Western blots), Northern blots, Southern blots, enzyme linked immunosorbent assay (ELISA), immunoprecipitation, immunofluorescence, flow cytometry, immunohistochemistry, nucleic acid hybridization techniques, nucleic acid reverse transcription methods, nucleic acid amplification methods, nucleic acid sequencing methods, and any combination thereof.

In certain embodiments, the step of detecting the presence or absence of Blastocystis sp. in the fecal sample is carried out by polymerase chain reaction (PCR), in particular by real-time quantitative polymerase chain reaction (qPCR).

In certain embodiments, detecting the presence or absence of Blastocystis sp. in the fecal sample by qPCR comprises steps of:
(a) performing an amplifying step comprising contacting the fecal sample, or a nucleic acid isolated therefrom, with a set of primers to produce an amplification product if a nucleic acid from Blastocystis sp. is present in the sample;
(b) performing a hybridizing step comprising contacting the amplification product with a detectable probe; and
(c) detecting the presence of the amplification product, wherein the presence of the amplification product is indicative of the presence of Blastocystis sp. in the fecal sample, and the absence of the amplification product is indicative of the absence of Blastocystis sp. in the fecal sample, wherein the set of primers to produce an amplification product from Blastocystis sp. comprises:
forward primers having the sequences set forth in:

SEQ ID NO: 1
(5′-TTTACTGTGAGAAAATTAGAGTGTTCAAAGC-3′),
and
SEQ ID NO: 2
(5′-TTTACTGTGAGAAAATTAGAGTGTTTAAAGC-3′),

and
reverse primers having the sequences set forth in:

SEQ ID NO: 3
(5′-TGCTTTCGCACTTGTTCATC-3′),
SEQ ID NO: 4
(5′-TGCTTTCGCACTAGTTCATC-3′),
and
SEQ ID NO: 5
(5′-TGCTTTCGCACCTGTTCATC-3′);

and a detectable probe having the sequence set forth in:

SEQ ID NO: 6
(5′-CAGTTGGGGGTATTCATATTCA-3′),

In certain embodiments, the detectable probe of SEQ ID NO: 6 comprises at least one locked nucleotide analog. In particular, the locked nucleotide analog may be a locked thymine analog. Preferably, the detectable probe of SEQ ID NO: 6 comprises one or more locked nucleotide analogs at any one of positions: 13, 14, 17, 19 and 20 in SEQ ID NO: 6.

In certain preferred embodiments, the detectable probe of SEQ ID NO: 6 comprises a locked thymine analog, in particular a LNA monomer with a thymine base, at each of positions 13, 14, 17, 19 and 20 in SEQ ID NO: 6.

In certain embodiments, the detectable probe is labelled at the 5′-end with the donor fluorescent moiety, FAM, and at the 3′-end with the quencher Iowa Black® FQ.

In certain embodiments, the set of primers to produce an amplification product from Blastocystis sp. and the detectable probe are comprised in a kit.

In certain embodiments, the method further comprises a step of: detecting by qPCR an exogenous internal positive control (IPC), the bacteriophage lambda, using a forward primer having the sequence set forth in SEQ ID NO: 7 (5′-GGACGTATCATGCTGGCCAA-3′), a reverse primer having the sequence set forth in SEQ ID NO: 8 (5′-GGAAATAGCCTCCGGCTCA-3′), and a detectable probe having the sequence set forth in SEQ ID NO: 9 (5′-TCCTTCGTGATATCGGACGTTGGCTG-3′) and labelled at the 5′-end with the donor fluorescent moiety, HEX, and at the 3′-end with the quencher Iowa Black® FQ and further labelled with the additional quencher ZEN positioned 6 to 15 bases from the 5′ donor fluorescent moiety, for example 6, 7, 8, 9, 10, 11, 12, 13, 14 or 15 bases from the 5′ donor fluorescent moiety, in particular 9 bases from the 5′ donor fluorescent moiety.

In certain embodiments, the kit mentioned above further comprises the forward primer having the sequence set forth in SEQ ID NO: 7, the reverse primer having the sequence set forth in SEQ ID NO: 8, and the detectable probe having the sequence set forth in SEQ ID NO: 9.

In another aspect, the present invention provides a method for improving management of a dairy herd, the method comprising steps of:
(1) predicting productive longevity in a dairy cow at the heifer stage using a method described above; and
(2) if, in step (1), the heifer is predicted to have a higher productive longevity, selecting the heifer as a replacement heifer for the dairy herd.

In certain embodiments, the method further comprises a step of:
(3) performing artificial insemination of the heifer when the heifer is ready for breeding.

In certain embodiments, the method further comprises a step of:
(4) if, in step (1), the heifer is predicted to have a lower productive longevity, culling the heifer from the dairy herd.

In certain embodiments, improving management of a dairy cow comprises, or results in, at least one of:
optimizing dairy herd renewal;
mitigating greenhouse gas emissions by the dairy herd and decreasing its carbon footprint;
decreasing veterinary costs for the dairy herd; and
improving profitability of the dairy herd.

In yet another aspect, the present invention provides a kit for use in the detection of the presence or absence of Blastocystis sp. in a fecal sample from a heifer or for improving management of a dairy herd, the kit comprising:
forward primers having the sequences set forth in SEQ ID NO: 1 and SEQ ID NO: 2;
reverse primers having the sequences set forth in SEQ ID NO: 3, SEQ ID NO: 4 and SEQ ID NO: 5, and
a detectable probe having the sequence set for in SEQ ID NO: 6 as defined above.

In certain embodiments, the kit further comprises a set of primers and probe for the detection of an exogenous internal positive control, the bacteriophage lambda, by PCR, comprising:
a forward primer having the sequence set forth in SEQ ID NO: 7;
a reverse primer having the sequence set forth in SEQ ID NO: 8; and
a probe having the sequence set forth in SEQ ID NO: 9 as defined above.

In certain embodiments, the kit further comprises instructions to perform a method according to the present invention.

In yet another aspect, the present invention provides the use of a kit as defined above for the detection of the presence or absence of Blastocystis sp. in a fecal sample from a heifer or for improving management of a dairy herd.

These and other objects, advantages and features of the present invention will become apparent to those of ordinary skill in the art having read the following detailed description of the preferred embodiments.

### BRIEF DESCRIPTION OF THE DRAWING

FIG. 1: Fluorescence plot (A) and standard curve of the TaqMan™ qPCR assay disclosed herein (B). A negative control was added in the fluorescence plot. Standard curve: CT=−3.86×log (conc)+41.41. Slope: −3.86; Y-intercept: 41.41; PCR efficiency calculated from slope: 0.82; and r2 of standard curve: 0.998. The standard curve was prepared with 10-fold serial dilutions of Blastocystis ST4 plasmids. Dilution points ranging from 10×10⁸ copies to 10×10³ copies were run in triplicate. Each plotted point represents the mean Ct value with standard error mean.

FIG. 2. Difference in days in milk production for the two categories of dairy cows for which Blastocystis biomarker status was identified when they were heifers.

FIG. 3. Difference in days in milk production for the two categories of culled dairy cows for which Blastocystis biomarker status was identified when they were heifers.

FIG. 4. Difference in α-diversity indices (Phylogenetic diversity, Faith's PD) between Blastocystis-positive heifers and Blastocystis-negative heifers.

FIG. 5. Difference in relative abundances of Escherichia-Shigella genera (d_Bacteria; p_Proteobacteria; c_Gammaproteobacteria; o_Enterobacterales; f_Enterobacteriace ae; g_Escherichia-Shigella) between animals carrying the Blastocystis biomarker and those lacking it.

### DETAILED DESCRIPTION OF CERTAIN PREFERRED EMBODIMENTS

As mentioned above, the present invention provides a biomarker, Blastocystis sp., the presence of which, in a fecal sample of a dairy cow at the heifer stage, is indicative of a future high productive longevity. The presence of the biomarker is also indicative of a richer and more diverse gastrointestinal microbiota, as well as an improved carbon footprint compared to a Blastocystis-negative dairy cow. The invention also concerns a qPCR method and primers and probes for detecting Blastocystis sp. in fecal samples obtained from heifers. Also provided are methods and kits for using the biomarker and/or the qPCR assay for predicting productive longevity in heifers, and for improving dairy herd management.

### I—Blastocystis sp.

In one aspect, the present invention provides the identity of an intestinal eukaryote, Blastocystis sp., the presence of which in a fecal sample of a dairy cow at the heifer stage was found to be indicative of a higher microbiota α-diversity, of a future higher productive longevity and of a lower carbon footprint compared to a Blastocystis-negative dairy cow.

Blastocystis sp. is a cosmopolitan protozoan that colonizes the gastrointestinal tract of humans and of a wide variety of animals worldwide (Nemati et al., J. Water Health, 2021, 19:687-704; Jiménez et al., Parasites Vectors, 2019, 12:376; Rauff-Adedotun et al., Biology, 2021, 10:990). This anaerobic microorganism is transmitted by the fecal-oral route through direct contact with infected hosts or through the consumption of food or water contaminated with cysts (Tan et al., Clin. Microbiol., Rev., 2008, 21:639-665). Human Blastocystis sp. infection can be associated with a variety of digestive symptoms, but the pathogenicity of this protozoan remains controversial in humans since the vast majority of individuals colonized by Blastocystis sp. do not present intestinal manifestations (Scanlan et al., FEMS Microbiol. Ecol., 2014, 90:326-330; Andersen et al., J. Clin. Microbiol., 2016, 54:524-528; Stensvold et al., Parasitol. Int., 2016, 65:763-771).

Interestingly, Blastocystis sp. colonization is not usually associated with disease in either wild or domestic animals (Hublin et al., Res. Vet. Sci., 2021, 135:260-282). In particular, cattle infected with Blastocystis sp. are healthy carriers. A recent meta-analysis (Shams et al., Comp. Immunol. Microbiol. Infect. Dis., 2021, 76:101650) has highlighted a 24.4% (95% CI: 16.9-33.9) prevalence for Blastocystis sp. in cattle after reviewing 29 international studies. Out of the 29 international studies, 25 relied on molecular detection of the protozoan. They found the prevalence of Blastocystis sp. in domestic dairy/beef cattle to vary between 2.9% and 100% (mean: 31.8%), depending on the country.

A wide genetic diversity of Blastocystis sp. has been identified in isolates obtained from birds and mammals based on nucleotide polymorphism at the small subunit ribosomal RNA (SSU rRNA) gene that has allowed the establishment of different subtypes (STs) (Stensvold et al., Trends Parasitol., 2020, 36:229-232; Maloney et al., Microorganisms, 2021, 9:997). At least 28 STs, considered as valid, are known to colonize avian and mammalian hosts with various frequencies (Stensvold et al., Trends Parasitol., 2020, 36:229-232; Higuera et al., Front. Vet. Sci., 2021, 8:732129; Maloney et al., Microorganisms, 2021, 9:1343; Jiménez et al., Parasit Vect., 2019, 12:1-9). Among these STs, ST1-ST4 account for more than 90% of the human infections reported globally (Stensvold et al., Int. J. Parasitol., 2009, 39:473-479), while ST5-ST8, ST10, ST12, ST14, and ST16 were rarely reported to be present in human stool, and are considered to be of animal origin (Alfellani et al., Acta Trop., 2013, 126:11-18; Ramírez et al., Infect. Genet. Evol., 2016, 41:32-35; Khaled et al., Microorganisms, 2020, 8:1408; Khaled et al., Microorganisms, 2021, 9, 184; Osorio-Pulgarin et al., Biology 2021, 10:669). In farm animals, a combination of zoonotic and enzootic subtypes has been reported (Hublin et al., Res Vet Sci., 2021, 135:260-282). For example, zoonotic subtypes, ST1-ST7 and ST12, and enzootic subtypes, ST10, ST14, ST17, ST21, ST23-ST26, have been found in cattle (Hublin et al., Res Vet Sci., 2021, 135:260-282; Santin et al., Parasitol Res., 2011, 109:205-212; Suwanti et al., Vet World., 2020, 13:231; Audebert et al., Parasitologia, 2022, 2:45-53), with subtypes ST10 and ST14 representing the most widely distributed subtypes in cattle.

Thus, the terms “Blastocystis sp.” and “Blastocystis”, which are used herein interchangeably, refer to an anaerobic protist belonging to a highly diversified Stramenopile phylum that can colonize the gastrointestinal tract of humans and various other species, including mammals and birds. In the context of the present invention, the terms “Blastocystis sp.” and “Blastocystis” more specifically refer to Blastocystis living in the gastrointestinal tract of cattle, and more particularly in the gastrointestinal tract of dairy cattle (Bos taurus). As indicated above, such Blastocystis may be of any subtype found in cattle, i.e., of a subtype selected from any one of subtypes ST1, ST2, ST3, ST4, ST5, ST6, ST7, ST10, ST12, ST14, ST17, ST21, ST23; ST24, ST25, and ST26.

### II—Detection of Blastocystis sp.

The methods of the present invention comprise a step of: detecting the presence or absence of Blastocystis sp. in a fecal sample obtained from a dairy cow to be tested, wherein the dairy cow is at the heifer stage.

### A. Dairy Cow at the Heifer Stage

The term “heifer”, as used herein, has its art understood meaning and refers to a nulliparous bovine female, i.e., a bovine female that has not given birth to a calf. By contrast, the term “cow”, as used herein, refers to a female bovine that has produced at least one calf. The term “dairy cow” refers more specifically to a cow that is reared for its milk. The terms “a dairy cow at the heifer stage” and “a dairy cow when the dairy cow is a heifer” are used herein interchangeably.

Puberty in dairy heifers is a function of breed, age, and weight. Depending on the dairy breeds, heifers reach puberty at between 30% and 60%, in particular between 40% and 60%, of the average mature weight. At this time, the hormonal patterns that regulate the estrous cycle begin developing and result in the heifer coming into heat on a regular basis. The first few heats may be erratic and anovulatory (no ovulation). After a couple of heats, heifers should cycle every 20-21 days (range 18-24 days). Heifers are generally at the proper weight and size for breeding at around 12-15 months of age. This allows heifers to calve when they are around 22-24 months old. However, the tradition of calving heifers around three years of age (36 months) is still practiced on some farms.

Thus, in a method described herein, the step of detecting the presence or absence of Blastocystis sp. in a fecal sample obtained from a dairy cow at the heifer stage is carried out when the heifer is 6 months old or older and 36 months (3 years) old or younger. In certain embodiments, a method described herein is carried out when the heifer is 6 months old or older and 24 months (2 years) old or younger. Preferably, the heifer to be tested is between 6 months and 20 months old, or between 6 months and 18 months old. For example, the heifer may be about 6, about 7, about 8, about 9, about 10, about 11, about 12, about 13, about 14, about 15, about 16, about 17, about 18, about 19, about 20, about 21, about 22, about 23 or about 24 months old.

The term “about”, as used herein and throughout the present document, in reference to a number, generally includes numbers that fall within a range of 10% in either direction of the number (greater than or less than the number) unless otherwise stated or otherwise evident from the context (except where such number would exceed 100% of a possible value).

The methods described herein may be applied to detect the presence or absence of Blastocystis sp. in a heifer belonging to any dairy cattle breed. As used herein, the term “cattle breed” refers to a group of cattle having common ancestors and/or sharing certain distinguishable traits that are not shared with cattle of other breeds. Those skilled in the art are familiar with cattle breed standards and/or characteristics.

Examples of suitable dairy cattle breeds include, but are not limited to, Abondance (originated from France), Alderney (from England), American Milking Devon (from the United States), Australian Bradford (from Australia), Australian Friesian (from Australia), Australian Lowline (from Australia), Australian Milking Zebu (from Australia), Ayrshire (from Scotland), Belgian Red (from Belgium), Bianca Modenese (from Italy), Brown Swiss (from Switzerland), Burlina (from Italy), Buša (from the Balkans), Canadienne (from Canada), Dairy Shorthorn (from England), Danish Jersey (from Denmark), Danish Red (from Denmark), Dexter (from Ireland), Estonian Red (from Estonia), Fleckvieh (from Austria), French Simmental (from France), German Black Pied Dairy (from Germany), Girolando (from Brazil), Guernsey (from Guernsey), Harzer Rotvieh (from Germany), Hays Converter (from Canada), Herens (from Switzerland), Holstein-Friesian (from the Netherlands), Illawarra Shorthorn (from Australia), Irish Moiled (from Ireland), Jamaica Hope (from Jamaica), Jersey (from Jersey, France), Lakenvelder (from the Netherlands), Meuse-Rhine-Issel (from Germany), Montbéliarde (from France), Normande (from France), Norwegian Red (Norway), Old Gloucester (from England), Randall Lineback (from the United States), Pie Rouge des Plaines (from France), Pinzgauer (from Austria), Red Chittagong (from Bangladesh), Red Poll (from England), Red Sindhi (from Pakistan), Sahiwal (from Pakistan), Sussex (from England), Tipo Carora (from Venezuela), Tyrol Grey (from Austria), and Vorderwald (from Germany).

In certain embodiments, the methods described herein are applied to heifers belonging to dairy cattle breeds known to be high milk producers. Examples of such dairy cattle breeds include, but are not limited to, Holstein-Friesian, Montbéliarde, Normande, Jersey, Brown Swiss, Guernsey, Ayrshire, and Milking Shorthorn.

### B. Fecal Sample

In a method described herein, the presence or absence of Blastocystis sp. is detected in a fecal sample obtained from the dairy heifer to be tested.

As used herein, the term “fecal sample” refers to a waste product from a heifer's digestive tract. The term “fecal sample” also encompasses any material derived by processing the fecal sample originally obtained. Derived materials include, but are not limited to, cells (or their progeny) isolated from the fecal sample, as well as proteins or nucleic acid molecules extracted from the fecal sample. Processing of a fecal sample may involve one or more of: concentration, dilution, filtration, sonication, homogenization, freezing and thawing, isolation, extraction, inactivation of interfering components, addition of reagents, and the like. In some embodiments, the fecal sample can be treated with a chemical reagent and/or biological reagent. Chemical and/or biological reagents can be employed to protect and/or maintain the stability of the sample, or nucleic acids comprised therein, during processing and/or storage. In addition, or alternatively, chemical and/or biological reagents can be employed to release nucleic acids from components of the fecal sample.

In the context of the present invention, the fecal sample may be obtained using any suitable method known in the art. In certain embodiments, the fecal sample is collected directly from the rectum of the heifer to be tested, as well known in the art. Appropriate disposable gloves should be worn, and the collected sample should be placed in a clean container and refrigerated at 4° C. Depending on the method used for detecting Blastocystis sp., the sample may be stored at 4° C. until parasitological examination, or frozen at −20° C. for antigen or PCR testing, or for next-generation sequencing.

In certain embodiments, the stools to be tested are preserved using potassium chromate before DNA extraction for PCR analysis. For example, a solution of 2.5% of potassium dichromate in water in a ratio of 1 volume to 1 volume of feces is used for preservation, after which feces are washed before DNA extraction, and PCR testing.

In certain embodiments, the step of detecting Blastocystis sp. in a fecal sample obtained from a heifer is performed on the fecal sample without any major manipulation of the sample.

In other embodiments, the detecting step is performed after in vitro culture.

In yet other embodiments, the detecting step is performed on microbial nucleic acid molecules extracted from the fecal sample, in particular on microbial genomic DNA. Methods of DNA extraction are well known in the art (see, for example, J. Sambrook et al., “Molecular Cloning: A Laboratory Manual”, 1989, 2^{nd} Ed., Cold Spring Harbour Laboratory Press: New York). There are also numerous versatile, commercially available kits that can be used to extract microbial nucleic acids (DNA or RNA) from biological samples, including fecal samples. Examples of such kits include, but are not limited to, the NucleoSpin® 96 Soil Kit, the NucleoSpin® Soil Mini Kit, and the NucleoSpin® DNA Stool (Macherey-Nagel GmbH & Co KG, Düren, Germany), the QIAamp® DNA Stool Minikit (Qiagen, Venlo, The Netherlands), the ZR Fecal DNA Kit (Zymo Research, United States), the Maxwell® RSC Fecal Microbiome DNA Kit (Promego, France), the E.Z.N.A® Stool DNA Kit (VWR-Omega Bio-Tek Inc.), the GenElute™ Stool DNA Isolation Kit (Sigma-Aldrich), the Stool DNA Isolation Kits (NorGen Biotek Corp.), the HigherPurity™ Stool DNA Extraction Kit (Canvax Biotech), and the Presto™ Stool DNA Extraction Kit (Geneaid Biotech Ltd.)

User Guides that describe in great detail the protocol to be followed are usually included in all these kits. Sensitivity, processing time and cost may be different from one kit to another. One of ordinary skill in the art can easily select the kit(s) most appropriate for a particular situation.

### C. Detection of Blastocystis sp.

In the context of the present invention, the step of detecting the presence or absence of Blastocystis sp. in a fecal sample obtained from a heifer may be performed using any suitable method. For example, such a method may be selected from the group consisting of microscopic examination, in vitro culture, molecular detection, and any combination thereof.

### 1. Microscopic Examination of Blastocystis sp.

In certain embodiments, the presence or absence of Blastocystis sp. in a fecal sample obtained from a dairy heifer is determined by light microscopic examination. For example, the analysis may be performed by observation of a smear stool diluted in saline solution at ×400 magnification. However, even for an experienced technician, Blastocystis polymorphism makes its identification difficult, which reduces the sensitivity of this analytical approach. Indeed, the Blastocystis vacuolar form, which is easily detectable, is not predominant in the stools while the dominant cystic form is difficult to distinguish from stool debris.

It is possible to precede this analysis with any known sedimentation technique, such as the classic formalin/ether (ethyl acetate) stool concentration. The formalin-ether concentration technique takes advantage of the high specific gravity of protozoan cysts compared to water. Their natural tendency to settle out in aqueous solutions can be accelerated by light centrifugation. It is worth noting that this technique is effective for cystic forms but not for vacuolar forms which can be lysed by the aggressiveness of the treatment.

Fecal smear staining may also improve the sensitivity of the observation method by direct light microscopy. Suitable staining agents include, but are not limited to, Lugol's iodine and trichrome (Wheatley, Am. J. Clin. Pathol., 1951, 21 (10): 990-991; Stensvold et al., Diagnostic Microbiology and Infectious Disease, 2007, 59:3:303-307; Termmathurapoj et al., Parasitology Research, 2004, 93:6:445-447). Other examples of suitable staining agents include Merthiolate-Iodine-Formaldehyde solution, Iron-Hematoxylin stain, and Giemsa stain.

### 2. In Vitro Culture of Blastocystis sp.

In certain embodiments, the analysis by microscopy follows a step of in vitro culture. This method has a high sensitivity, but is time-consuming and laborious. Furthermore, it should be noted that, in some instances, culture allows preferential growth of one Blastocystis subtype over another if more than one subtype is present in the fecal sample.

Methods of in vitro microbial culture from fecal samples are known in the art. For example, in such a culture method, approximately 50-100 mg of the fecal sample are subjected to culture in Jones' medium supplemented with 10% horse serum and antibiotics (penicillin, streptomycin). Samples are incubated under anaerobic conditions at 37° C. in an incubator. The growth of Blastocystis and the distinct morphological and reproductive stages can then be confirmed by microscopic observation of culture at 48 hours and 72 hours of incubation using Lugol's iodine staining and light microscopy at 400×.

### 3. Molecular Detection of Blastocystis sp.

Numerous studies have compared the effectiveness of different methods for detecting Blastocystis sp. (Bart et al., BMC Infect. Dis., 2013, 13:389; Dogruman-Al et al., PLOS One, 2010, 5 (11): e15484; Kumarasamy et al., Parasit. Vectors., 2014, 7:162; Osman et al., PLOS Negl. Trop. Dis., 2016, 10 (3): e0004496; Poirier et al., J. Clin. Microbiol., 2011, 49 (3): 975-983; Roberts et al., Am. J. Trop. Med. Hyg., 2011, 84 (2): 308-312; Santos and Rivera, Asian Pac. J. Trop. Dis., 2013, 6 (10): 780-784; Stensvold et al., Diag. Microbiol. Infect. Dis., 2007, 59 (3): 303-307; Stensvold et al., J. Clin. Microbiol., 2012, 50 (6): 1847-1851; Tan, Clin. Microbiol. Rev., 2008, 21 (4): 639-665; Hublin et al., Res. Vet. Sci., 2021, 135:260-282). All have clearly demonstrated the greater sensitivity of molecular methods compared to morphological and cultural methods.

Thus, in certain preferred embodiments, the step of detecting the presence or absence of Blastocystis sp. in a fecal sample obtained from a heifer is carried out using a molecular method. Any of a wide variety of molecular methods may be used in the context of the present invention. Examples of suitable molecular methods include, but are not limited to, immunoblots (Western blots), Northern blots, Southern blots, enzyme linked immunosorbent assay (ELISA), immunoprecipitation, immunofluorescence, flow cytometry, immune-histochemistry, nucleic acid hybridization techniques, nucleic acid reverse transcription methods, nucleic acid amplification methods, nucleic acid sequencing methods and any combination thereof.

a. Immunodiagnostic Methods

In certain embodiments, the molecular method used to detect the presence or absence of Blastocystis sp. in a fecal sample obtained from a heifer is an immunodiagnostic method. The development of commercial and in-house immunodiagnostic methods, either to detect parasite antigens or host antibodies, has allowed to increase the sensitivity and specificity of the detection of protozoan infection. Immunodiagnostic tests are generally inexpensive, user-friendly, and enable fast-obtained results. Such commercial tests have been developed for the detection of Blastocystis sp. Examples include, but are not limited to, the ParaFlor B™ antibody kit (Boulder Diagnostics, Boulder, CO, USA), which detects Blastocystis by immunofluorescence and which was used in dairy cattle (Fayer et al., Parasitology Research, 2012, 111:1349-1355); the CoproELISA Blastocystis™ kit (Sayon Diagnostics, Ashdod, Israel), an ELISA test for the detection of Blastocystis-specific antigens in stool (Dogruman-Al et al., Parasitology Research, 2015, 114:495-500), and the Blasto-Fluor™ kit (Antibodies Inc., Davis, CA, USA), an immunofluorescence antibody (IFA) stain specific for Blastocystis (Dogruman-Al et al., PLOS ONE, 2010, 5 (11): e15484). Other examples of ELISA kits include the Human Blastocystis Hominis ELISA kit and the Blastocystis spp. ELISA kit, which are used for the analysis of human stool samples.

b. Polymerase Chain Reaction (PCR)

In other embodiments, the step of detecting the presence or absence of Blastocystis sp. in a fecal sample obtained from a heifer is a PCR (Polymerase Chain Reaction) assay. Over the past decades, the development of molecular methods to diagnose intestinal parasite has been centered on PCR assays. This was the case with the RFLP (Restriction Fragment Length Polymorphism) and RAPD (Random Amplification of Polymorphic DNA) methods developed for the detection of Blastocystis (Böhm-Gloning et al., Tropical Medicine & International Health, 1997, 2 (8): 771-778; Hisao and Ayako, Int. J. Parasitol., 1996, 26 (10): 1111-1114; Tan et al., Parasitol. Research, 2006, 99 (4): 459-465; Yoshikawa et al., Parasitol. Research, 2004, 92 (1): 22-29), even if these approaches are rarely used today since they do not provide sequence data.

State-of-the art detection of Blastocystis now relies on real-time PCR (qPCR) using genomic DNA extracted from fecal samples. The small subunit of ribosomal RNA (SSU rRNA) gene is currently a popular gene marker used for accurate identification and subtyping of Blastocystis sp. (Tan, Clin. Microbiol. Rev., 2008, 21:639-665). For SSU IRNA sequence, the barcode region is a valid representation of the whole gene and the barcode sequence of the primers cover all polymorphic positions, ensuring that no phylogenetic signal information is lost (Abdo et al., J. Parasit. Dis., 2021, 45:738-745; Adao et al., Ann. Parasitol., 2016, 62:193-200). Moreover, the short barcode sequences can be used in publicly available sequence databases that can render consensus ST nomenclature and allele assignments per barcode sequence query much more convenient than using full SSU IRNA sequences (Adao et al., Ann. Parasitol., 2016, 62:193-200). Examples of qPCR methods for the detection of Blastocystis sp. include, but are not limited to, the qPCR assays described in Poirier et al., J. Clin. Microbiol., 2011, 49 (3): 975-983; Stensvold et al., J. Clin. Microbiol., 2012, 50 (6): 1847-1851; Dagci et al., Iran J. Parasitol., 2014, 9 (4): 519-529; and WO 2021/094661.

There are also multiplex qPCRs that make it possible to amplify Blastocystis, among other microorganisms, including, but are not limited to, Roche LightMix® Gastro Parasites multiplex PCR assay (Roche Diagnostics) (Friesen et al., Clin Microbiol Infect., 2018, 24 (12): 1333-1337); and Allplex® GI parasite (Seegene) (Argy et al., Parasite, 2022, 29:5).

The present Inventors have developed a TaqMan™ 5′-nuclease real-time PCR (TaqMan™ qPCR) including an internal process control (IPC) for the detection of Blastocystis and applicable to genomic DNAs extracted from fecal samples obtained from heifers. The developed assay enables successful amplification of DNAs from all subtypes of Blastocystis known in the art, and especially all Blastocystis subtypes described in cattle (Bos taurus) (see below).

c. Next-Generation Amplicon Sequencing

In other embodiments, the step of detecting the presence or absence of Blastocystis sp. in a fecal sample obtained from a heifer is carried out using next-generation sequencing (NGS). As used herein, the term “next-generation sequencing” refers to a number of different technologies that have the capacity to sequence oligonucleotides at speeds above those possible with conventional sequencing methods (e.g., the Sanger sequencing method), due to performing and reading out thousands to millions of sequencing reactions in parallel. They have opened a whole new set of applications in microbiology. In particular, next-generation sequencing technologies have been broadly applied to various microbiome studies, including the detection of mixed infections of parasites and the identification of rare or novel subtypes of parasitic infection.

Many sequencing platforms employing next-generation sequencing have been developed. The workflow of library preparation, sequencing, and raw data output are similar for all platforms. The only difference among these platforms is the sequencing methods, which are known as pyrosequencing, sequencing by synthesis, sequencing by ligation, and ion semiconductor sequencing. Examples of next-generation sequencing platforms/systems include, but are not limited to, Massively Parallel Signature Sequencing (Lynx Therapeutics); 454-pyro-sequencing (454 Life Sciences/Roche Diagnostics); solid-phase, reversible dye-terminator sequencing (Solexa/Illumina); Sequencing by Oligonucleotide Ligation and Detection (SOLID) technology (Applied Biosystems); ion semiconductor sequencing (ION Torrent); DNA nanoball sequencing (Complete Genomics); and technologies available from Pacific Biosciences, Intelligent Bio-systems, Oxford Nanopore Technologies, and Helicos Biosciences.

Next-generation sequencing has been used for detecting Blastocystis sp. in fecal samples obtained from humans (Rojas-Velázquez et al., Parasit. Vectors, 2019, 12:566; Castañeda et al., Parasit. Vectors, 2020, 13:521; Cinek et al., Parasit. Vectors, 2020, 14:399) as well as from animals including domestic animals including pigs, minipigs, cows, dogs, horses, goats, sheep and llama (Higuera et al., Front Vet Sci., 2021, 8:732129); in deers (Maloney et al., Microorganisms, 2021, 9 (6): 1343); cattle (Abarca et al., Vet Sci., 2021, 8 (9): 191); bird species (Maloney et al., Parasite Epidemiol. Control, 2020, 9: e00138); rats (Defaye et al., Sci. Rep., 2020, 10:9146); swine (Ramesh et al., Sci. Rep., 2021, 11:16994); and horses (Baek et al., Microorganisms, 2022, 10 (9): 1693).

Generally, the sequencing primers used in a next-generation sequencing method can comprise portions compatible with the selected method. Next-generation sequencing technologies and the constraints and design parameters of associated sequencing primers are well known in the art (see, e.g., Shendure et al., Nature, 2008, 26 (10): 1135-1145; Mardis, Trends in Genetics, 2007, 24 (3): 133-141; Su, et al., Expert. Rev. Mol. Diagn., 2011, 11 (3): 333-343; Zhang et al., J. Genet. Genomics, 2011, 38 (3): 95-109; Nyren et al., Anal. Biochem., 1993, 208:17175; Bentley, Curr. Opin. Genet. Dev., 2006, 16:545-552; Strausberg et al., Drug Disc Today, 2008, 13:569-577).

4. qPCR Method of Detection of Blastocystis sp.

As mentioned above, the present Inventors have developed a TaqMan™ real-time quantitative PCR (TaqMan™ qPCR) assay, which includes an internal process control (IPC), for the detection of Blastocystis sp. in fecal samples obtained from heifers. More specifically, the TaqMan™ qPCR assay was designed to detect a part of gene coding for the small subunit ribosomal RNA (SSUrRNA) of Blastocystis sp. The terms “real-time quantitative PCR” and “qPCR”, which are used herein interchangeably, have their art understood meaning and refer to a technique that simultaneously amplifies and quantifies target nucleic acids using PCR, wherein the quantification is by virtue of an intercalating fluorescent dye or sequence-specific probes which contain fluorescent reporter molecules that are only detectable once hybridized to a target nucleic acid. The term “TaqMan™ qPCR” refers to a qPCR assay involving polymerase chain reaction technology and 5′-exonuclease activity of Taq polymerase. A TaqMan™ qPCR assay makes use of a TaqMan™ probe. As used herein, the term “TaqMan™ probe” refers to an oligonucleotide (designed to anneal within a PCR amplified product) in which fluorescent materials (fluorophores) acting as a reporter and a quencher are attached to both ends of the oligonucleotide probe, e.g., a fluorescent reporter at the 5′ end and a quencher at the 3′ end.

Thus, in certain embodiments, the step of detecting the presence or absence of Blastocystis sp. in a fecal sample obtained from a dairy heifer is carried out by real-time polymerase chain reaction (qPCR) and comprises steps of:
(a) performing an amplifying step comprising contacting the fecal sample, or a nucleic acid isolated therefrom, with a set of primers to produce an amplification product if a nucleic acid from Blastocystis sp. is present in the sample;
(b) performing a hybridizing step comprising contacting the amplification product with a detectable probe; and
(c) detecting the presence of the amplification product, wherein the presence of the amplification product is indicative of the presence of Blastocystis sp. in the fecal sample, and the absence of the amplification product is indicative of the absence of Blastocystis sp. in the fecal sample.

One skilled in the art knows how to perform an amplifying (or amplification) step (as defined in step (a) of the developed TaqMan™ qPCR method). As used herein, the term “amplification” refers to a method or process that increases the representation of a population of specific nucleic acid sequences in a sample. An amplification reaction is carried out in amplification conditions, i.e., in conditions that promote annealing and extension of primer sequences. Such conditions are well-known in the art. For example, in a PCR reaction, amplification conditions generally comprise thermal cycling, i.e., cycling of the reaction mixture between two or more temperatures. Amplification conditions encompass all reaction conditions including, but not limited to, temperature and temperature cycling, amplification reaction reagents, ionic strength, pH, and the like. As used herein, the term “amplification reaction reagents” refers to reagents used in nucleic acid amplification reactions and may include, but are not limited to, buffers; enzymes having exonuclease activity; enzyme cofactors such as magnesium or manganese; salts; and deoxynucleotide triphosphates (dNTPs) such as deoxyadenosine triphosphate (dATP), deoxyguanosine triphosphate (dGTP), deoxycytidine triphosphate (dCTP), and deoxythymidine triphosphate (dTTP). Amplification reaction reagents may readily be selected by one skilled in the art depending on the amplification method used.

In the context of the present invention, the amplifying step (step (a)) is carried out in the presence of a polymerase enzyme having 5′ to 3′ nuclease activity.

The amplifying step is carried out in the presence of a set of primers to produce an amplification product if a nucleic acid from Blastocystis sp. (i.e., the target sequence) is present in the sample. The terms “primer” and “amplification primer” are used herein interchangeably. They refer to an isolated oligonucleotide which is capable of acting as a point of initiation of synthesis of a primer extension product that is a complementary strand of a portion of the target sequence, when placed under suitable amplification conditions in the presence of nucleotides and an agent for nucleic acid polymerization. In a TaqMan™ qPCR method described above, the set of primers comprises:
forward primers having the sequences set forth in:

SEQ ID NO: 1
(5′-TTTACTGTGAGAAAATTAGAGTGTTCAAAGC-3′),
and
SEQ ID NO: 2
(5′-TTTACTGTGAGAAAATTAGAGTGTTTAAAGC-3′),

reverse primers having the sequences set forth in:

SEQ ID NO: 3
(5′-TGCTTTCGCACTTGTTCATC-3′),
SEQ ID NO: 4
(5′-TGCTTTCGCACTAGTTCATC-3′),
and
SEQ ID NO: 5
(5′-TGCTTTCGCACCTGTTCATC-3′);

These primers were designed from an alignment of small subunit ribosomal RNA (SSUrRNA) sequences of Blastocystis subtypes ST1 and ST26. In order to ensure the amplification of all subtypes of Blastocystis described in the literature, and especially all Blastocystis subtypes described in cattle (Bos taurus), a mixture of one-nucleotide degenerated forward and reverse primers is used in the PCR set of primers.

One skilled in the art knows how to perform a hybridizing (or hybridization) step (as defined in step (b) of the developed TaqMan™ qPCR method). The term “hybridization” refers to the formation of complexes between nucleotide sequences which are sufficiently complementary to form complexes via Watson-Crick base pairing or non-canonical base pairing. It will be appreciated that hybridizing sequences need not have perfect complementarity to provide stable complexes (or hybrids), for example between a detectable probe and an amplification product. In many situations, stable hybrids will form where fewer than about 10% of the bases are mismatches. For examples of hybridization conditions and parameters see, e.g., J. Sambrook et al., “Molecular Cloning: A Laboratory Manual”, 2012, 4^{th} Ed., Cold Spring Harbor Press: Plainview, NY; F.M. Ausubel, “Current Protocols in Molecular Biology”, 1994, John Wiley & Sons: Secaucus, NJ.

The hybridizing step in the TaqMan™ qPCR according to the present invention is carried out in the presence of a detectable probe. The term “probe” refers to an oligonucleotide capable of selectively hybridizing to at least one portion of a target sequence under appropriate conditions (e.g., a portion of a target sequence that has been amplified, i.e., the amplification product). The terms “detectable”, labeled”, “labeled with a detectable agent” and “labeled with a detectable moiety” are used herein interchangeably. These terms are used to specify that an entity (e.g., an oligonucleotide probe) can be visualized, for example, following binding to another entity (e.g., an amplification product). Preferably, a detectable agent or moiety is selected such that it generates a signal which can be measured and whose intensity is related to the amount of bound entity.

TM probe In the context of the present invention, the PCR detectable probe is a TaqMan™ that is labeled at the 5′-end with a fluorescent moiety and at the 3′-end with a quencher moiety. Suitable fluorophores and quenchers for use with TaqMan™ probes are disclosed, for example in U.S. Pat. Nos. 5,210,015; 5,804,375; 5,487,792; and 6,214,979; and WO 01/86001 (each of which is incorporated herein by reference in its entirety). Examples of quenchers include, but are not limited to DABCYL (i.e., 4-(4′-dimethylaminophenylazo)-benzoic acid) succinimidyl ester, diarylrhodamine carboxylic acid, succinimidyl ester (or QSY-7), and 4′,5′-dinitrofluorescein carboxylic acid, succinimidyl ester (or QSY-33) (all available, for example, from Molecular Probes), quencher1 (Q1; available from Epoch Biosciences, Bothell, WA, USA), or “Black hole quenchers” BHQ-1, BHQ-2, and BHQ-3 (available from BioSearch Technologies, Inc., Novato, CA, USA), or Iowa Black FQ®, Iowa Black RQ®, TAO, ZEN™ (available from Integrated DNA Technologies, Coraville, IA, USA) and BBQ-650™ (available from Biosearch Technologies, Inc., Petaluma, CA, USA).

In a TaqMan™ qPCR method described above, the detectable probe has the sequence set forth in:

SEQ ID NO: 6
(5′-CAGTTGGGGGTATTCATATTCA-3′),

and is labelled in 5′ with a donor fluorescent moiety and in 3′ with a quencher.

In certain embodiments, the detectable probe of SEQ ID NO: 6 is a locked nucleic acid. As used herein, the term “locked nucleic acid” (or LNA) has its art understood meaning, and refers to an oligonucleotide that contains at least one locked nucleotide analog. The terms “locked nucleotide analog”, “locked nucleic acid monomer”, and “LNA monomer”, which are used herein interchangeably, refer to a nucleotide having a modified ribose moiety in which the ribose moiety comprises an extra bridge connecting the 2′ and 4′ carbons (U.S. Pat. Nos. 6,749,499; 6,734,291; and 6,670,461). When incorporated into an oligonucleotide probe, locked nucleic acid monomers impart heightened structural stability, resulting in increased hybridization melting temperature (Tₘ). In the context of the present invention, a locked analog of a particular nucleotide is designed to exhibit the same base-pairing specificity, e.g., a locked analog of a thymine (T) will base-pair with an adenine (A). For example, to retain the same base-pairing specificity, the base component of a locked analog of a particular nucleotide is chosen to be identical to the particular nucleotide, e.g., the locked analog of a thymine is a LNA monomer with a thymine base.

In certain embodiments, the detectable probe of SEQ ID NO: 6 comprises at least one locked nucleotide analog, for example, one, two, three, four, five, six, or seven locked nucleotide analogs. In certain preferred embodiments, the detectable probe of SEQ ID NO: 6 comprises one or more locked thymine analogs, for example, one, two, three, four, five, six, or seven locked thymine analogs. Preferably, a locked thymine analog is a LNA monomer with a thymine base. In certain embodiments, the one or more locked thymine analogs are located at one or more of positions: 13, 14, 17, 19 and 20 in SEQ ID NO: 6. In certain embodiments, the detectable probe of SEQ ID NO: 6 contains five locked thymine analogs at positions 13, 14, 17, 19 and 20 in SEQ ID NO: 6. Preferably, the five locked thymine analogs are LNA monomers with a thymine base.

In certain preferred embodiments, the detectable probe having the sequence SEQ ID NO: 6 (whether it comprises locked nucleotide analogs or not) is labelled in 5′ with the donor fluorescent moiety, FAM (6-carboxyfluorescein), and in 3′ with the quencher Iowa Black® FQ (IABKFQ).

One skilled in the art knows how to detect the presence or absence of a product amplified using a TaqMan™ qPCR method. In the context of the present invention, the detection of fluorescence is indicative of the presence of Blastocystis sp. in the fecal sample tested, while the absence of fluorescence is indicative of the absence of Blastocystis in the fecal sample.

In certain preferred embodiments, the TaqMan™ qPCR assay according to the present invention includes an exogenous internal positive control (IPC). Such control is used to detect false negatives, and qualitatively detect the presence of amplification inhibitory substances in a sample. Thus, in certain embodiments, a method according to the present invention further comprises detecting by qPCR an exogenous IPC, the bacteriophage lambda, using a set of primers comprising:
a forward primer having the sequence set forth in:

SEQ ID NO: 7
(5′-GGACGTATCATGCTGGCCAA-3′),

and
a reverse primer having the sequence set forth in:

SEQ ID NO: 8
(5′-GGAAATAGCCTCCGGCTCA-3′),

and
a detectable probe having the sequence set forth in:

SEQ ID NO: 9
(5′-TCCTTCGTGATATCGGACGTTGGCTG-3′),

wherein the detectable probe is labelled in 5′ with a donor fluorescent moiety and in 3′ with a quencher. In certain preferred embodiments, the detectable probe used in the IPC detection is labelled in 5′ with the donor fluorescent moiety, HEX (Hexachloro-Fluorescein), and in 3′ with the quencher Iowa Black® FQ (IABKFQ). In certain preferred embodiments, the detectable probe having the sequence set forth in SEQ ID NO: 9 is further labelled with an additional quencher, ZEN, positioned 6 to 15 bases from the 5′ donor fluorescent moiety, for example 6, 7, 8, 9, 10, 11, 12, 13, 14 or 15 bases from the 5′ donor fluorescent moiety, in particular 9 bases from the 5′ donor fluorescent moiety. As known in the art, the presence of an additional quencher at such a position results in a reduction of the background fluorescence and an increased sensitivity.

As known in the art, the forward and reverse primers and detectable probes may be used in any suitable concentrations. One skilled in the art knows how to determine suitable concentrations. For example, a sample of 2 μL of extracted DNA may be added to a qPCR mixture, wherein the forward primer of SEQ ID NO: 1 is present at a concentration comprised between about 500 and about 800 nM, the forward primer of SEQ ID NO: 2 at a concentration comprised between about 10 and about 200 nM, the reverse primer of SEQ ID NO: 3 at a concentration comprised between about 100 and about 500 nM, the reverse primer of SEQ ID NO: 4 at a concentration comprised between about 150 and about 550 nM, the reverse primer of SEQ ID NO: 5 at a concentration comprised between about 20 and about 250 nM, the Blastocystis detectable probe of SEQ ID NO: 6 at a concentration comprised between 200 and 600 nM, the IPC forward primer of SEQ ID NO: 7 at a concentration comprised between 200 and 650 nM, the IPC reverse primer of SEQ ID NO: 8 at a concentration comprised between 300 and 700 nM, and the IPC detectable probe of SEQ ID NO: 9 at a concentration comprised between 100 and 400 nM.

A dairy heifer identified as being infected with Blastocystis sp. using a method according to the present invention may be characterized as “Blastocystis-positive”. In contrast, when a method according to the present invention concludes that the fecal sample provided by a dairy heifer does not contain Blastocystis sp., the dairy heifer is characterized as “Blastocystis-negative”.

### III—Uses of the Inventive Methods

As will be appreciated by those of ordinary skill in the art, Blastocystis sp. colonization in a dairy heifer, which has been found to be indicative of a future longer productive lifespan, of a richer and more diverse gastrointestinal microbiota, as well as an improved carbon footprint compared to a Blastocystis-negative dairy heifer, can be used as a biomarker. As used herein, the term “biomarker” refers to a substance that is a distinctive indicator of a biological process, biological event and/or pathological condition.

### A. Method of Predicting Productive Longevity in a Dairy Cow

Thus, the present invention provides a method for predicting productive longevity in a dairy cow, the method comprising a step of: detecting the presence or absence of Blastocystis sp. in a fecal sample obtained from the dairy cow when the dairy cow is a heifer, wherein the presence of Blastocystis sp. in the fecal sample is indicative of a higher productive longevity in the dairy cow, and the absence of Blastocystis sp. in the fecal sample is indicative of lower productive longevity in the dairy cow.

The term “productive longevity”, as used herein to characterize a dairy cow, refers to the time period spanning from the beginning of the first lactation cycle to the end of the last lactation cycle or to the event associated with the end of the animal's production (death or culling of the animal). The term “lactation cycle” is the period between one calving and the next. Thus, the productive longevity, which is used herein interchangeably with “productive life or lifespan”, includes the dry periods between lactation cycles (i.e., the periods during which the cow does not produce any milk). The productive longevity is expressed using a time unit. For example, it may be expressed in days, or weeks, or months.

In a method according to the present invention, the presence of Blastocystis sp. in the fecal sample obtained from a dairy heifer is predictive of a future high productive longevity, i.e., of a higher productive longevity, or of an increased produced longevity, compared to a Blastocystis-negative dairy cow. The present Inventors have shown that, on average, Holstein-Friesian dairy cows having completed their lactation career and having been identified, at the heifer stage, as Blastocystis-positive, have a productive longevity that is around 138 days longer compared to non-infected Holstein-Friesian dairy cows, which corresponds to an improvement of around 30% in productive longevity. It will be understood by one skilled in the art that the increase in productive longevity of Blastocystis-positive dairy cows compared to Blastocystis-negative dairy cows may be cattle breed-dependent.

Thus, as used herein the term “higher productive longevity” refers to a productive lifespan of a Blastocystis-positive dairy cow that is at least about 10 days longer than the productive lifespan of a Blastocystis-negative dairy cow of the same cattle breed. For example the higher productive longevity may be at least about 20 days longer, at least about 30 days longer, at least about 40 days longer, at least about 50 days longer, at least about 60 days longer, at least about 70 days longer, at least about 80 days longer, at least about 90 days longer, at least 100 days longer, at least about 110 days longer, at least about 120 days longer, at least about 130 days longer, at least about 140 days longer, at least about 150 days longer, or more than 150 days longer, for example, about 160 days, about 170 days, about 180 days, about 190 days, about 200 days, about 210 days, about 220 days, about 230 days, about 240 days, about 250 days, about 260 days, about 270 days, about 280 days, about 290 days, about 300 days, about 310 days, etc., The term “higher productive longevity” may also refer to a productive lifespan of a Blastocystis-positive dairy cow that is at least about 2% longer than the productive lifespan of a Blastocystis-negative dairy cow of the same cattle breed. For example, the higher productive longevity may be at least about 4% longer, at least about 6% longer, at least about 8% longer, at least about 10% longer, at least about 12% longer, at least about 14% longer, at least about 16% longer, at least about 18% longer, at least about 20% longer, at least about 21% longer, at least about 22% longer, at least about 23% longer, at least about 24% longer, at least about 25% longer, at least about 26% longer, at least about 27% longer, at least about 28% longer, at least about 29% longer, at least about 30% longer, or more than 30% longer, for example about 32%, about 34%, about 36%, about 38%, about 40%, about 45%, about 50%, about 55%, about 60%, about 65% longer, etc., In certain embodiments, the term “higher productive longevity” refers to a productive lifespan of a Blastocystis-positive dairy cow that comprises one additional lactation cycle compared to the productive lifespan of a Blastocystis-negative dairy cow of the same cattle breed.

As used herein the term “lower productive longevity” is the exact opposite of the term “higher productive longevity”, as defined above. In other words, the term “lower productive longevity” refers to a productive lifespan of a Blastocystis-negative dairy cow that is at least about 10 days shorter than the productive lifespan of a Blastocystis-positive dairy cow of the same cattle breed. For example the lower productive longevity may be at least about 20 days shorter, at least about 30 days shorter, at least about 40 days shorter, at least about 50 days shorter, at least about 60 days shorter, at least about 70 days shorter, at least about 80 days shorter, at least about 90 days shorter, at least 100 days shorter, at least about 110 days shorter, at least about 120 days shorter, at least about 130 days shorter, at least about 140 days shorter, at least about 150 days shorter, or more than 150 days shorter, for example, about 160 days, about 170 days, about 180 days, about 190 days, about 200 days shorter, etc., The term “lower productive longevity” may also refer to a productive lifespan of a Blastocystis-negative dairy cow that is at least about 2% shorter than the productive lifespan of a Blastocystis-positive dairy cow of the same cattle breed. For example, the lower productive longevity may be at least about 4% shorter, at least about 6% shorter, at least about 8% shorter, at least about 10% shorter, at least about 12% shorter, at least about 14% shorter, at least about 16% shorter, at least about 18% shorter, at least about 20% shorter, at least about 21% shorter, at least about 22% shorter, at least about 23% shorter, at least about 24% shorter, at least about 25% shorter, at least about 26% shorter, at least about 27% shorter, at least about 28% shorter, at least about 29% shorter, at least about 30% shorter, or more than 30% shorter, for example about 32%, about 34%, about 36%, about 38%, about 40%, about 45%, about 50%, about 55%, about 60%, about 65% longer, etc., In certain embodiments, the term “lower productive longevity” refers to a productive lifespan of a Blastocystis-negative dairy cow that comprises one lactation event fewer compared to the productive lifespan of a Blastocystis-positive dairy cow of the same cattle breed.

### B. Method of Predicting Carbon Footprint of a Dairy Cow

It will be understood by one skilled in the art that a dairy cow with a longer productive lifespan emits less methane (CH₄) in the environment per unit of milk produced when compared to a dairy cow with a shorter productive lifespan. Heifers and cows that have not completed their first lactation have particularly unfavorable results in terms of their methane emission per unit of product. The present Inventors have calculated that Blastocystis-positive Holstein-Friesian dairy cows have a 17.5% lower carbon contribution per kg of milk produced compared to Blastocystis-negative animals of the same cattle breed.

Thus, the present invention provides a method for predicting carbon footprint of a dairy cow, the method comprising a step of: detecting the presence or absence of Blastocystis sp. in a fecal sample obtained from the dairy cow when the dairy cow is a heifer, wherein the presence of Blastocystis sp. in the fecal sample is indicative of a lower carbon footprint for the dairy cow, and the absence of Blastocystis sp. in the fecal sample is indicative of higher carbon footprint for the dairy cow.

The term “carbon footprint”, as used herein when applied to a dairy cow, refers to the carbon dioxide equivalent of the amount of greenhouse gases (mostly methane) produced by the dairy cow over its lifetime divided by the amount of milk it produces over the same period of time. Carbon dioxide equivalence uses CO₂ as a basis to establish the potential impact of emission. A carbon dioxide equivalent, or CO₂ equivalent (CO₂-eq), is a metric measure used to compare the emissions from various greenhouse gases on the basis of their global-warming potential (GWP), by converting amounts of other gases to the equivalent amount of carbon dioxide with the same global warming potential. GWP is 1 for CO₂, it is 25 for methane (CH₄).

The term “lower carbon footprint”, as used herein, refers to a carbon footprint of a Blastocystis-positive dairy cow that is at least about 2% lower than the carbon footprint of a Blastocystis-negative dairy cow of the same cattle breed. For example, the lower carbon footprint may be at least about 3% lower, at least about 4% lower, at least about 5% lower, at least about 6% lower, at least about 7% lower, at least about 8% lower, at least about 9% lower, at least about 10% lower, at least about 11% lower, at least about 12% lower, at least about 13% lower, at least about 14% lower, at least about 15% lower, at least about 16% lower, at least about 17% lower, at least about 18% lower, at least about 19% lower, at least about 20% lower, or more than 20% lower that the carbon footprint of a Blastocystis-negative dairy cow of the same cattle breed.

As used herein, the term “higher carbon footprint” is the exact oppositive to the term “lower carbon footprint” defined above.

### C. Method of Predicting Microbiota α-Diversity

In another aspect, the present invention provides a method for predicting gut microbiota α-diversity in a dairy cow, the method comprising a step of: detecting the presence or absence of Blastocystis sp. in a fecal sample obtained from the dairy cow when the dairy cow is a heifer, wherein the presence of Blastocystis sp. in the fecal sample is indicative of a higher microbiota α-diversity in the dairy cow, and the absence of Blastocystis sp. in the fecal sample is indicative of lower microbiota α-diversity in the dairy cow.

As used herein, the term “microbiota α-diversity” has its art understood meaning. It summarizes the distribution of species abundances in a given sample of gut microbiota into a single number that depends on species richness and evenness. Diversity indices measure the overall community heterogeneity.

The term “higher microbiota α-diversity”, as used herein, refers to a gut microbiota α-diversity of a Blastocystis-positive dairy cow that is at least about 5% higher than the gut microbiota α-diversity of a Blastocystis-negative dairy cow of the same cattle breed. For example, the higher microbiota «-diversity may be at least about 5% higher, at least about 6% higher, at least about 7% higher, at least about 8% higher, at least about 9% higher, at least about 10% higher, at least about 11% higher, at least about 12% higher, at least about 15% higher, at least about 16% higher, at least about 17% higher, at least about 18% higher, at least about 19% higher, at least 20% higher, or more than 20% higher than the microbiota cx-diversity of a Blastocystis-negative dairy cow of the same cattle breed.

As used herein, the term “lower microbiota α-diversity” is the exact oppositive of the term “higher microbiota α-diversity”, defined above.

### D. Method of Improving Management of a Dairy Herd

As will be appreciated by those of ordinary skill in the art, a biomarker whose presence at an early stage in a dairy cow's life correlates with significantly greater gut microbiota richness and diversity as well as with future higher productive longevity and lower carbon footprint can be used to help dairy farmers gather crucial information on individual heifers and plan for the future of their herds. Indeed, having this data on hand when a dairy cow is at the heifer stage allows to make more accurate, strategic breeding and replacement decisions.

Thus, the present invention provides a method for improving management of a dairy herd, the method comprising steps of: (1) predicting productive longevity in a dairy cow at the heifer stage using a method described herein; and (2) if in step (1), the heifer is predicted to have a higher productive longevity, selecting the heifer as a replacement heifer for the dairy herd.

As used herein, the term “replacement heifer” refers to a heifer that is selected to be bred and eventually placed in the dairy herd, generally to replace a culled dairy cow.

### 1. Artificial Insemination

A method for improving management of a dairy herd, as defined above, will generally further comprise a step of: (3) performing artificial insemination on the replacement heifer when the replacement heifer is ready for breeding.

As used herein, the term “artificial insemination”, refers to a technique in which sperm is collected from a bull, processed, stored and manually introduced into the female reproductive tract at an appropriate time for the purpose of conception. Artificial insemination has become one of the most imperative techniques for the genetic improvement of farm animals since preferably the semen from genetically superior sires are used to inseminate the female animals artificially. Artificial insemination has a range of benefits to dairy farmers, such as providing the best genetics, supporting animal health and ensuring the safety of people who work on farms.

With conventional artificial insemination, there is a 50/50 chance of male or female offspring but, in the dairy industry, female offspring are more desirable than male offspring. Dairy farmers use sexed semen to increase the numbers of heifer calves born and reduce the number of unwanted male dairy calves. Thus, in certain embodiments, the replacement heifer is artificially inseminated with sexed semen. The term “sexed semen”, as used herein, has its art understood meaning, and refers to a semen that has gone through the process of sorting X-chromosome bearing sperm cells and Y-chromosome bearing sperm cells to produce progenies of a desired sex either male or female with almost 80-90% accuracy. In the context of the present invention, the replacement heifer is artificially inseminated with sexed semen to produce a heifer calf.

Artificial insemination is performed when the replacement heifer is ready for breeding. The term “when ready for breeding”, as used herein, refers not only to a time when the heifer has reached puberty and is considered mature to breed, but also to the appropriate time for insemination during the heifer's estrous cycle. One skilled in the art of dairy farming knows how to determine whether and when a heifer is ready for breeding based on age, weight, and/or regular estrous cycle. Determination of the optimal time for insemination generally involves heat detection, which aims to identify heifers that are about to ovulate. Different methods, including methods focusing on physical, behavioral and/or physiological signs that are associated with estrus (heat), are known in the art. Highest conception rates for artificial insemination generally occurs between 4 and 12 hours after the onset of estrus.

### 2. Optimization of the Mating Process

Heifers carrying the biomarker have a better potential for productive longevity. To allow the realization of this potential, optimized mating can be considered: for example, the use of semen doses from bulls with favorable genetic or genomic indexes for birth conditions (Birth Ease, Calving Ease). Knowledge of biomarker status can be integrated into mating management software. This information may influence the choice of bull genetics (based on their breed and/or genetic or genomic indexes) chosen to be used differentially with heifers carrying or not carrying the biomarker.

### 3. Culling

A method for improving management of a dairy herd, as defined above, will generally further comprise a step of: (4) if, in step (1), the heifer is predicted to have a lower productive longevity, culling the heifer from the dairy herd.

The term “culling”, as used herein, has its art understood meaning in the cattle management field and refers to the removal of a dairy cow from the herd. The dairy cow may be sold or sent to be slaughtered. Cows may be sold for “dairy” purposes, which means that these cows can start or continue their productive life on a different dairy farm. Generally, a dairy cow is culled from the herd due to mortality, disease (e.g., mastitis), low milk production, reproductive problems including infertility. Culling is a tool that dairy farms can use to improve their overall herds (by keeping the best cows and replacing the worst), to keep the herd size from growing beyond capacity, and to maximize profits. In the context of the present invention, the decision to cull a dairy cow may be made when the dairy cow is at the heifer stage, as early as when the heifer is predicted to have a lower productive longevity.

### 4. Dairy Herd Management

A method for improving management of a dairy herd, as defined above, may comprise, or may result in, or may be a method for, at least one of:
Optimizing dairy herd renewal;
Mitigating greenhouse gas emissions by the dairy herd and decreasing its carbon footprint;
Decreasing veterinary costs for the dairy herd; and
Improving profitability of the dairy herd.

Dairy animals with a longer productive lifespan are globally more efficient, bringing economic, environmental and animal welfare benefits.

To achieve a more efficient, and thus profitable herd, the dairy farmer needs to know exactly when to replace cows in the milking herd and cull them. To make this decision, the dairy farmer has to predict a cow's milk output in the next lactation, predict the value of the replacement heifer and compare those predictions. To be able to make a useful comparison, reliable data are needed. Thus, a method according to the present invention, which predicts future productive longevity in dairy heifers, allows to make more accurate and strategic replacement decisions, thereby optimizing dairy herd renewal. Indeed, by keeping only the heifers that will be the best milk cows, it is possible to reach or maintain the optimal size of the milking herd.

A milking herd with a higher average milk production per day will need less replacement heifers. This increases long-term profitability, as it results in lower rearing costs. Furthermore, this leads to an increase in the proportion of mature, high-producing cows in the herd.

A more efficient dairy herd also decreases carbon footprint. Indeed, a herd with a greater proportion of multiparous cows excretes less methane in the environment per unit of milk produced compared to a herd with a higher proportion of heifers and primiparous cows. It also results in less manure and thus less nitrogen and phosphorus that pollute water and air. By reaching or maintaining the optimal size of a milking herd, less barn space is needed, and feed efficiency, which is the amount of 3.5% fat-corrected milk produced per pound of feed dry matter consumed, is improved. In addition, a higher feed efficiency decreases the land needed for feed crops as well as feed transport and the corresponding fuel use.

Other expected benefits of a more efficient dairy herd include a reduction in veterinary costs due to fewer health treatments and performance-limiting health disorders. Indeed, heifers selected as replacement heifers according to a method of the present invention are Blastocystis-positive, which has been shown to be associated a significantly greater gastrointestinal microbiota richness and diversity and a drastic decrease in the presence of pathogens responsible for subclinical or clinical bovine mastitis, a disease which causes severe inflammation of the mammary gland and udder tissue in dairy cattle.

### IV—Kits

In another aspect, the present invention provides kits comprising materials useful for carrying out a detection method of the invention. The procedures described herein may be performed by analytical laboratories, research laboratories, or by practitioners (e.g., veterinarians). The invention provides kits that can be used in these different settings.

Materials and reagents for performing a detection method of the present invention may be assembled together in a kit.

In certain embodiments, the inventive kit for use in the detection of the presence or absence of Blastocystis sp. in a fecal sample obtained from a heifer, or for improving management of a dairy herd, comprises a set of primers comprising:
forward primers having the sequences set forth in:

SEQ ID NO: 1
(5′-TTTACTGTGAGAAAATTAGAGTGTTCAAAGC-3′),
and
SEQ ID NO: 2
(5′-TTTACTGTGAGAAAATTAGAGTGTTTAAAGC-3′),

and
reverse primers having the sequences set forth in:

SEQ ID NO: 3
(5′-TGCTTTCGCACTTGTTCATC-3′),
SEQ ID NO: 4
(5′-TGCTTTCGCACTAGTTCATC-3′),
and
SEQ ID NO: 5
(5′-TGCTTTCGCACCTGTTCATC-3′);

and a detectable probe having the sequence set forth in:

SEQ ID NO: 6
(5′-CAGTTGGGGGTATTCATATTCA-3′),

and labelled in 5′ with a donor fluorescent moiety and in 3′ with a quencher. As already described above, the detectable probe of SEQ ID NO: 6 may comprise at least one locked nucleotide analog.

In certain preferred embodiments, the detectable probe having the sequence SEQ ID NO: 6 is labelled in 5′ with the donor fluorescent moiety, FAM (6-carboxyfluorescein), and in 3′ with the quencher Iowa Black® FQ (IABKFQ).

In certain embodiments, the kit further comprises primers and probe to detect an exogenous IPC, wherein the set of primers comprises:
a forward primer having the sequence set forth in:

SEQ ID NO: 7
(5′-GGACGTATCATGCTGGCCAA-3′),

and
a reverse primer having the sequence set forth in:

SEQ ID NO: 8
(5′-GGAAATAGCCTCCGGCTCA-3′),

and
wherein the detectable probe has the sequence set forth in:

SEQ ID NO: 9
(5′-TCCTTCGTGATATCGGACGTTGGCTG-3′),

and is labelled in 5′ with a donor fluorescent moiety and in 3′ with a quencher.

In certain preferred embodiments, the detectable probe used in the IPC detection is labelled in 5′ with the donor fluorescent moiety, HEX, and in 3′ with the quencher Iowa Black® FQ (IABKFQ). Preferably, the detectable probe having the sequence set forth in SEQ ID NO: 9 is further labelled with an additional quencher, ZEN, positioned 6 to 15 bases from the 5′ donor fluorescent moiety, for example 6, 7, 8, 9, 10, 11, 12, 13, 14 or 15 bases from the 5′ donor fluorescent moiety, in particular 9 bases from the 5′ donor fluorescent moiety.

A kit may further comprise instructions for using the kit to perform a detection according to a method of the invention.

The kit may further comprise one or more of: extraction buffer and/or reagents, amplification buffer and/or reagents, hybridization buffer and/or reagents, labeling buffer and/or reagents, and detection means. Protocols for using these buffers and reagents to perform different steps of the detection procedure may be included in the kit.

The reagents may be supplied in a solid (e.g., lyophilized) or liquid form. The kits of the present invention may optionally comprise different containers (e.g., vial, ampoule, test tube, flask or bottle) for each individual buffer and/or reagent. Each component will generally be suitable as aliquoted in its respective container or provided in a concentrated form. Other containers suitable for conducting certain steps of the disclosed methods may also be provided. The individual containers of the kit are preferably maintained in close confinement for commercial sale.

Instructions for using the kit according to a method of the invention may comprise instructions for processing the fecal sample obtained from the heifer, instructions for extracting microbial DNA from the fecal sample, instructions for performing the test, and/or instructions for interpreting the results as well as a notice in the form prescribed by a governmental agency (e.g., FDA) regulating the manufacture, use or sale of pharmaceuticals or biological products.

Also provided herein is the use of a kit as described above for the detection of the presence or absence of Blastocystis sp. in a fecal sample obtained from a heifer, or for improving management of a dairy herd.

### EXAMPLES

The following examples describe some of the preferred modes of making and practicing the present invention. However, it should be understood that the examples are for illustrative purposes only and are not meant to limit the scope of the invention. Furthermore, unless the description in an Example is presented in the past tense, the text is not intended to suggest that experiments were actually performed, or data were actually obtained.

### Example 1: Development of a qPCR Assay for the Detection of Blastocystis sp.

The qPCR assay developed to detect Blastocystis sp. is based on the well-known TaqMan™ technology, and includes the use of an exogenous Internal Positive Control (IPC). The qPCR has been validated on a Rotor-Gene Q instrument (QIAGEN) but is transposable to other qPCR instruments such as the QuantStudio™ 3 Real-Time PCR System (Applied Biosystems).

The primers, primer pairs and probes used in the present study are presented in Table 1.

Primers and probe for Blastocystis amplification were designed from an alignment of small subunit ribosomal RNA (SSUrRNA) sequences of Blastocystis subtypes ST1 and ST26. The specificity of the Blastocystis primers was demonstrated using the Blast software (NCBI) and confirmed by the absence of qPCR amplification of other fecal parasites, thus showing that the test is specific for Blastocystis spp. In order to ensure the amplification of all subtypes of Blastocystis described in the literature, and especially all Blastocystis subtypes described in cattle (Bos taurus), a mixture of one-nucleotide degenerated forward and reverse primers was used in the PCR mix (see Table 1). Primers and probe positions listed in Table 1 are based on the Genbank Blastocystis reference U51151.

Thus, the forward primer, PF_BLASTO_725, was degenerated with a SNP at position 750 and the qPCR mix used contains between, 500 and 800 nM of the primer PF_BLASTO_725 and between 10 and 200 nM of the primer PF_BLASTO_725_2. The reverse primer, PR_BLASTO_924, was degenerated with a SNP at position 912 or at position 913 and the qPCR mix contains between 100 and 500 nM of the primer PR_BLASTO_924, between 150 and 550 nM of the primer PR_BLASTO_924_2 and between 20 and 250 nM of the primer PR_BLASTO_924_3. These primer pairs allow the amplification of a 200 bp PCR product.

TABLE 1
Sequences of primers and probes developed and used in the TaqMan ™ qPCR assay.
The percentages shown next to the Blastocystis forward and reverse primer sequences
represent the molar concentration proportions of primers used in the qPCR mix. SNPs,
as well as probe reporters and quenchers are shown in bold on the sequences.

| | | |
|---|---|---|
| | | SEQ |
| Primers/ | | ID |
| Probes | Sequences | NOs: |

Blastocystis

| | | |
|---|---|---|
| F primers | PF_Blasto_725: 5′-TTTACTGTGAGAAAATTAGAGTGTTCAAAGC-3′ (90%) | 1 |
| | PF_Blasto_725_2: 5′-TTTACTGTGAGAAAATTAGAGTGTTTAAAGC-3′ (10%) | 2 |
| R primers | PR_Blasto_924: 5′-TGCTTTCGCACTTGTTCATC-3′ (40%) | 3 |
| | PR_Blasto_924_2: 5′-TGCTTTCGCACTAGTTCATC-3′ (50%) | 4 |
| | PR_Blasto_924_3: 5′-TGCTTTCGCACCTGTTCATC-3′ (10%) | 5 |
| Probe | TP_Blasto_850_G: 5′-CAGTTGGGGGTATTCATATTCA-3′ | 6 |
| | Labelled in 5′ with the donor fluorescent moiety, FAM (6- | |
| | carboxyfluorescein), and in 3′ with the quencher Iowa Black ® FQ | |
| | (IABkFQ); and comprising at each of positions 13, 14, 17, 19 and 20, | |
| | a LNA monomer with a thymine base. | |

IPC

| | | |
|---|---|---|
| F primer | PF_IPC_For: 5′-GGACGTATCATGCTGGCCAA-3′ | 7 |
| R primer | PR_IPC_Rev: 5′-GGAAATAGCCTCCGGCTCA-3 | 8 |
| Probe | TP_IPC_14011_Y: 5′-TCCTTCGTGATATCGGACGTTGGCTG-3′ | 9 |
| | Labelled in 5′ with the donor fluorescent moiety, HEX (Hexachloro- | |
| | Fluorescein), and in 3′ with the quencher Iowa Black ® FQ (IABkFQ) | |
| | and further labelled with an additional quencher, ZEN, positioned 9 | |
| | bases from the 5′ donor fluorescent moiety. | |

The Blastocystis probe, named TP_Blasto_850_G, is a PrimeTime LNA® qPCR Probe.

The primers for IPC and the IPC probe were designed on the bacteriophage lambda genome. The pair of primers used for the qPCR consists of the forward IPC_qPCR_For and reverse IPC_qPCR_Rev primers. The probe for the IPC, named TP_IPC 14011 Y, is a PrimeTime qPCR probe with ZEN quencher.

The Blastocystis probe, named TP_Blasto_850_G, is a PrimeTime LNA® qPCR Probe.

As shown in Table 2, the positions of the primers and probe on the SSUrDNA sequence of Blastocystis differ from previously published qPCR primers and probe for Blastocystis SSUrDNA detection.

TABLE 2
Primers and probe sequences positions of the qPCR method described herein and
previously published PCR methods. The positions are based on the Genbank SSUrDNA
Blastocystis reference sequence U51151. (NGS: Next Generation Sequencing).

| | Positions of | Positions of Reverse | Positions of |
|---|---|---|---|
| | Forward Primer | Primer | TaqMan Probe |
| Present work | 725-755 | 905-924 | 850-865 |
| Poirier et al.ª | 22-44 | 334-353 | NA (Sybr Green) |
| Scicluna et al.^{b} | 5-24 | 590-611 | NA (end-point PCR) |
| Maloney et al.^{c} | 445-464 | 905-924 | NA (NGS) |
| Stensvold et al.^{d} | 1633-1655 | 1726-1751 | 1697-1722 |
| Dagci et al.^{e} | 590-613 | 922-949 | 850-870 |
| WO 2021/094661 | 300-322 | 364-381 | 328-357 |

^{a}Poirier et al., J. Clin. Microbiol., 2011, 49(3): 975-983.
^{b}Scicluna et al., Protist., 2006, 157(1): 77-85.
^{c}Maloney et al., Infect. Genet. Evol., 2019, 73: 119-125.
^{d}Stensvold et al., J. Clin. Microbiol., 2012, 50(6): 1847-1851.
^{e}Dagci et al., Iran J. Parasitol., 2014, 9(4): 519-529.

Determination of qPCR System Efficiency and Sensitivity:

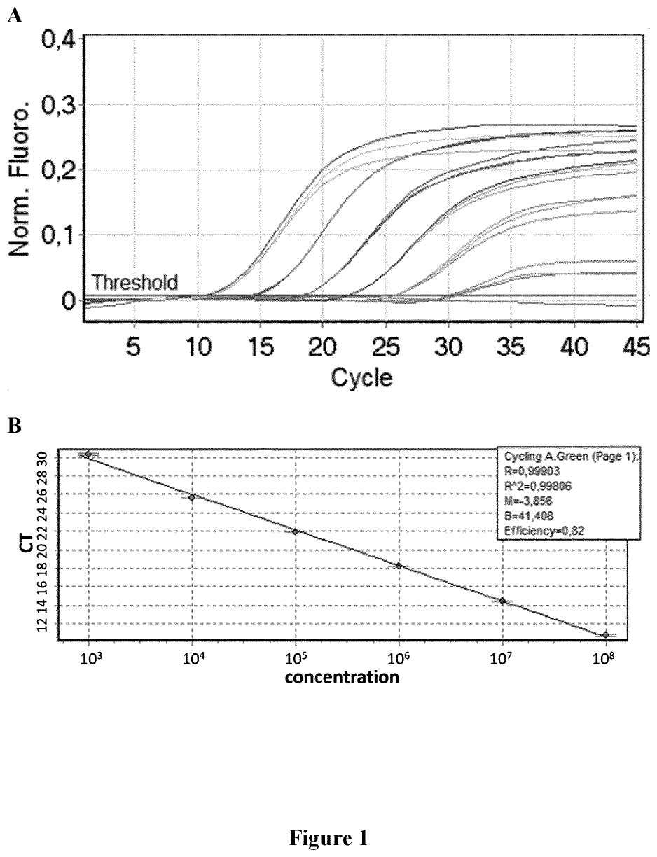

Evaluation of the efficiency and sensitivity of the qPCR assay disclosed herein was performed using a dilution range of Blastocystis ST4 plasmids. Dilution points ranging from 10×10⁸ copies to 10×10⁰ copies were run in triplicate. The standard curve obtained was used to determine the qPCR efficiency (FIG. 1).

The limit of detection of the qPCR was assessed to be 5×10¹ copies/μL of fecal sample DNA and the PCR efficiency was 0.82 (FIG. 1).

The TaqMan™ qPCR method disclosed herein was compared to one of the reference methods for molecular Blastocystis detection, which consists in a SYBR Green PCR (Sasaki et al., Anim. Sci. J., 2012, 83:95-102). The detection of Blastocystis in 894 fecal samples showed that the positive rate of the established TaqMan™ real-time PCR method was 81.8% (731/894), while the positive rate of the convention SYBR Green PCR (Poirier et al., J. Clin. Microbiol., 2011, 49 (3): 975-983) was 78.3% (700/894), with a coincidence rate of 73.6%. The high specificity, sensitivity, and rapidity of the TaqMan™ real-time PCR assay disclosed herein with the use of IPC to monitor for false negative results make this method highly suitable for the detection of the Blastocystis sp. in fecal samples from heifers.

### Example 2: Establishment of Blastocystis sp. In Heifers as a Biomarker of Higher Productive Longevity, Higher α-Diversity, and Lower Carbon Footprint

### 1. Description of the Cohort of the Study

With no selection bias, 200 nulliparous Holstein-Friesian cows (heifers) were followed throughout their milk production careers between 2017 and 2021. The animals belonged to 19 different herds.

Fecal microbiota samples were collected from these animals between 16 and 28 months of age. For fecal sampling, heifers were rectally finger-stimulated with sterile-gloved hands to facilitate the collection of a 25 g fecal sample. The samples were immediately refrigerated at +4° C. (for 1 to 2 hours) before being frozen and stored before analysis at −20° C.

A DNA extraction method was used to isolate and purify the DNA from these samples. More specifically, total genomic DNA was extracted from approximately 200 mg of animal fecal samples using the NucleoSpin 96 Soil Kit or NucleoSpin Soil Mini Kit (Macherey-Nagel GmbH & Co KG, Düren, Germany) according to the manufacturer's recommendations. A qPCR assay according to the invention was used to identify the presence or absence of the Blastocystis biomarker in this complex environment.

The results obtained are presented in Table 3.

Based on the presence or absence of the Blastocystis biomarker, it was possible to determine two categories of heifers: carriers (n=120) and non-carriers (n=80) of the biomarker. Thus, the Blastocystis biomarker was present in 60% of the heifers of the study.

TABLE 3
Prevalence of the Blastocystis biomarker in nulliparous cows
in the different studied herds.

| | total | number of carriers/ | percentage of carriers/ |
|---|---|---|---|
| | number | number of non- | percentage of |
| Herd | of heifers | carriers | non-carriers |
| 59_1 | 17 | 13/4 | 76.47%/23.53 % |
| 59_2 | 6 | 3/3 | 50%/50% |
| 59_3 | 20 | 10/10 | 50%/50% |
| 59_4 | 16 | 8/8 | 50%/50% |
| 59_5 | 6 | 5/1 | 83.33%/16.67% |
| 59_6 | 2 | 1/1 | 50%/50% |
| 59_7 | 6 | 4/2 | 66.67%/33.33% |
| 59_8 | 3 | 3/0 | 100%/0% |
| 59_9 | 1 | 0/1 | 0%/100% |
| 59_10 | 20 | 6/14 | 30%/70% |
| 59_11 | 17 | 15/2 | 88.24%/11.76% |
| 59_12 | 6 | 5/1 | 83.33%/16.67% |
| 59_13 | 1 | 1/0 | 100%/0% |
| 59_14 | 9 | 8/1 | 88.89%/11.11% |
| 62_1 | 21 | 18/3 | 85.71%/14.29% |
| 62_2 | 18 | 5/13 | 27.78%/72.22% |
| 62_3 | 21 | 11/10 | 52.38%/47.62% |
| 62_4 | 10 | 4/6 | 40%/60% |
| TOTAL | 200 | 120/80 | 60%/40% |

### 2. Association Between the Biomarker Occurrence and Productive Longevity in Dairy Cattle

a. Effect on the Whole Cohort

Productive longevity is defined here as the difference between the end of lactation and the first lactation event, as defined by Sasaki et al. (Anim. Sci. J., 2012, 83:95-102). Longevity is a relatively difficult trait to select for in dairy cattle breeding because of its low heritability and the numerous factors that influence longevity in dairy cattle. Longevity and productive longevity may be the consequence of several factors:
i. intrinsic factors: lactation, health, conformation traits, and reproductive performance (Ferris et al., J. Dairy Sci., 2014, 97:5206-5218);
ii. external factors: milk price, nutrition, management, policy, feed costs (Vries and Marcondes, Animal, 2020, 14: s155-s164).

According to their Blastocystis biomarker status, the two groups of heifers were found to exhibit significantly different average results for productive longevity (see Table 4).

TABLE 4
Productive longevity based on Blastocystis status.

| | carriers | non-carriers |
|---|---|---|
| Duration (in days) of productive life | 721.14 days | 646.56 days |

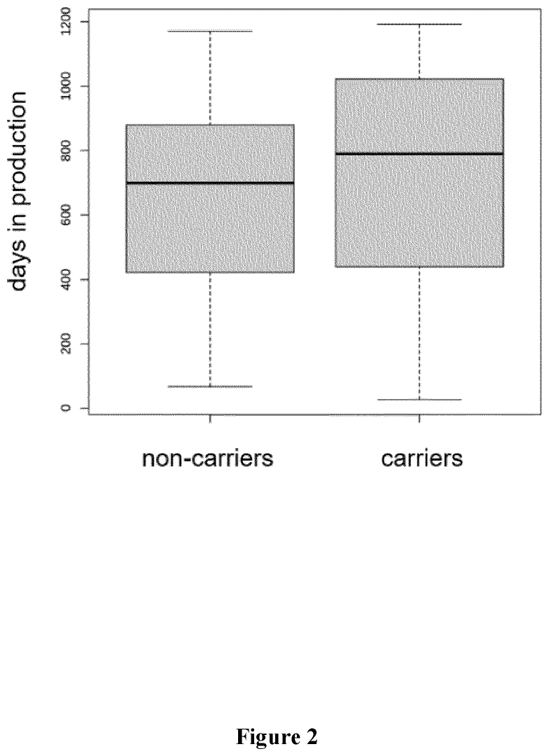

On average, Blastocystis heifers identified as carrying the biomarker had a 74.60 day-longer productive longevity (i.e., an 11.54% improvement in productive longevity when taking non-carrier heifers as a reference-Table 4). A non-parametric Wilcoxon test was performed, resulting in a p-value of 0.04123 (FIG. 2).

It should be noted that, in this determination, some of the animals studied were still in lactation. It was therefore decided to carry out a different determination using only the animals with a known date of end of lactation (i.e., cows that have completed their lactation career).

b. Effect on Nulliparous Animals Having Completed their Lactation Career and Belonging to Herds with at Least 12 Characterized Animals.

The results are presented in Table 5.

TABLE 5
Prevalence of the Blastocystis biomarker in
nulliparous heifers that have been culled
(i.e., with an of lactation period) in herds
containing more 12 of such heifers.

| | Number of | number | percentage of |
|---|---|---|---|
| | heifers having | of carriers/ | carriers/ |
| | completed their | number of | percentage |
| Herd | lactation career | non-carriers | of non-carriers |
| 59_1 | 13 | 10/3 | 76.92%/23.08% |
| 59_3 | 13 | 8/5 | 61.54%/38.46% |
| 62_1 | 14 | 11/3 | 78.57%/21.43% |
| 62_2 | 13 | 5/8 | 38.46%/61.54% |
| 62_3 | 14 | 6/8 | 42.86%/57.14% |
| TOTAL | 67 | 40/27 | 59.70%/40.30% |

This dataset shows a similar prevalence of the Blastocystis biomarker as the first one shown in Table 3.

According to their Blastocystis biomarker status, the two groups of heifers having completed their lactation career were found to exhibit significantly different average results for productive longevity (see Table 6).

TABLE 6
Productive longevity (not truncated) as a function
of Blastocystis status.

| | carriers | non-carriers |
|---|---|---|
| Duration (in days) | 607.85 days | 469.93 days |
| of productive life | | |

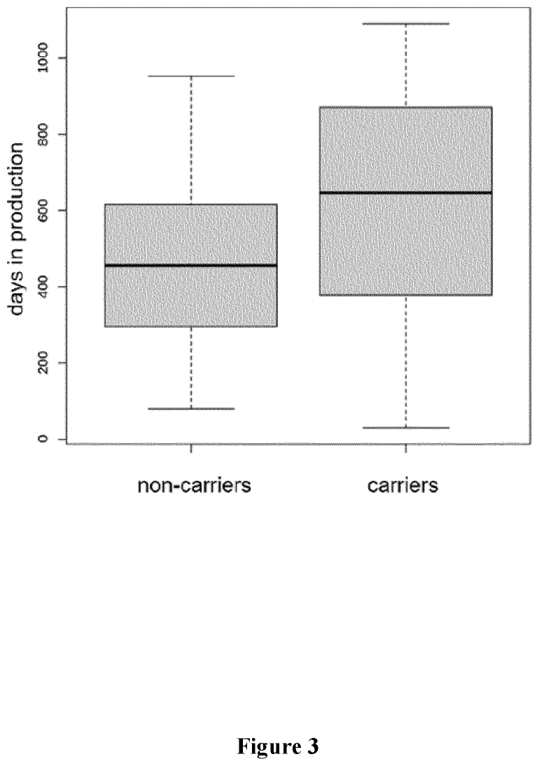

On average, heifers having completed their lactation career and carrying the Blastocystis biomarker were found to have a 137.92 day longer longevity (i.e., an improvement of +29.35% in productive longevity when considering heifers not carrying the biomarker). A Student's t-test shows the significant difference between these two averages (p-value=0.04711, see FIG. 3).

### 3. Association Between the Blastocystis Biomarker Occurrence and Milk Production

a. Milk Per Day of Life in Milk Production

The results obtained are shown in Table 7 below.

TABLE 7
Average milk quantity per day of productive life
according to Blastocystis status.

| | carriers | non-carriers |
|---|---|---|
| kg of milk produced per | 23.81 kg | 23.76 kg |
| day of productive life | | |

The presence of the biomarker does not affect the milk yield of animals identified as Blastocystis carriers at the heifer stage as there is no statistically significant difference between the two averages of milk quantity per day of productive life (Table 7).

b. Cumulative Milk Produced During a Complete Career as a Function of Biomarker Status Per Day of Life in Milk Production

The results obtained are shown in Table 8 below.

TABLE 8
Average of cumulative milk production (kg/life) over a
lifetime as a function of Blastocystis status.

| | carriers | non-carriers |
|---|---|---|
| kg of milk produced during | 14 791.58 kg | 11 384.59 kg |
| productive career | | |

On average, an animal identified as a Blastocystis carrier at the heifer stage was found to produce an additional 3 406.98 kg of milk during her life without degrading her daily performance (Tables 7 and 8).

c. Analysis of Milk Constituent as a Function of Blastocystis Biomarker

No significant differences were observed between the two groups of animals carrying or not the biomarker for: fat, protein content or leukocyte concentrations. The Blastocystis biomarker status of the animals did not influence the intrinsic qualities of the milk produced.

### 4. Association Between the Blastocystis Biomarker and Methane Production Over a Lifetime

Methane emission intensity and profitability of dairy cows are more favorable for cows with long productive lives, while cows that have not completed their first lactation have particularly unfavorable results in terms of their emissions per unit of product, and rearing costs were generally not paid back (Grandl et al., Animal, 2019, 13 (1): 198-208).

The last decades have shown that with the increase of milk yields in cows, their productive life clearly decreased in a concomitant way (Knaus, J. Sci. Food and Agriculture, 2009, 89:1107-1114). Several studies show that an increase in productive life span could be considered a good option for mitigating Greenhouse Gas (GHG) emissions because it reduces the emissions associated with raising replacement animals (Zehetmeier et al., Animal, 2012, 6:154-166; Bell et al., J. Agric. Sci., 2015, 153:138-151), such a strategy would be correlated with better profitability (De Vries, J. Dairy Sci., 2017, 100:4184-4192; Horn et al., Organic Agriculture, 2012, 2:127-143). Assuming an average amount of methane produced per day of life of 406 g/day (Vanlierde et al., J. Dairy Sci., 2018, 101 (8): 7618-7624), and assuming a multiplicative factor of 25 as carbon equivalent (Table 9), a comparison of carbon footprint is possible between animals carrying or not the Blastocystis biomarker.

TABLE 9
Global warming power (GWP) for the 6 gases or gas families covered
by the Kyoto Protocol (Source: IPCC, 4^{th} Assessment Report, 2007).

| Gaz | Formula | GWP/CO₂ |
|---|---|---|
| Carbon dioxide | CO₂ | 1 |
| Methane | CH₄ | 25 |
| Nitrous oxide | N₂O | 298 |
| Perfluorocarbons | CₙF₂ₙ₊₂ | 7400 à 12200 |
| Hydrofluorocarbons | CₙHₘFₚ | 120 à 14800 |
| Sulfur hexafluoride | SF₆ | 22800 |

TABLE 10
Elements to establish the carbon balance between animals
carrying or not the Blastocystis biomarker.

| | carriers | non-carriers |
|---|---|---|
| kg of milk produced during | 14 791.58 kg | 11 384.59 kg |
| productive career | | |
| average age in days at the | 1500.23 days | 1360.51 days |
| end of lactation | | |
| kg of methane produced | 609.09 kg | 552.37 kg |
| (including the growing phase) | | |
| ratio kg of methane/kg of milk | 0.0412 | 0.0485 |
| carbon equivalent of the ratio | 1.03 | 1.21 |
| kg of methane/kg of milk | | |

The presence of the Blastocystis biomarker is associated with greater productive longevity of the dairy cow. Animals carrying the biomarker, having an improved longevity, produce more milk during their career while not degrading their daily milk yield as previously demonstrated. Thus, these animals with greater productive longevity more easily reimburse their ecological debt by amortizing the heifer phase (phase during which the animal does not produce milk and produces greenhouse gases) over a longer period. Animals not carrying the Blastocystis biomarker have a 17.5% higher carbon contribution per kg of milk produced compared to animals with the biomarker (Table 10).

### 5. Link Between the Blastocystis Biomarker Presence and Fecal Microbiota Parameters

### a. α-Diversity

The α-diversity metric is associated with the complexity of a given microbiota. At the fecal microbiota level, high microbiota richness and diversity are considered beneficial, as they improve the microbiota stability, especially under nutritionally challenged conditions, and allow it to use limiting resources more efficiently (Russell et al., Science, 2001, 292 (5519): 1119-1122). In ruminants, a number of metabolic disorders, such as subacute and acute ruminal acidosis, are associated with a reduction in rumen and hindgut bacterial diversity (Khafipour et al., Appl. Environ. Microbiol., 2009, 75 (22): 7115-7124; Azad et al., “Composition of rumen microbiota alters following diet-induced milk fat depression in dairy cows”, Joint Annual Meeting of American Society of Animal Science and American Dairy Science Association, Orlando, Fl., Jul. 12-16, 2015; Plaizier et al., Front Microbiol., 2017, 7: Article 2128).

Therefore, α-diversity and richness indices can be used as biomarkers to determine health or disease status of the animal (Khafipour et al., Animal Frontiers, 2016, 6 (2): 13-19). DNA samples used here to detect the presence or absence of the Blastocystis biomarker were also used to perform a metagenetic analysis targeting an ubiquitous bacterial marker (i.e., the 16S small ribosomal subunit gene).

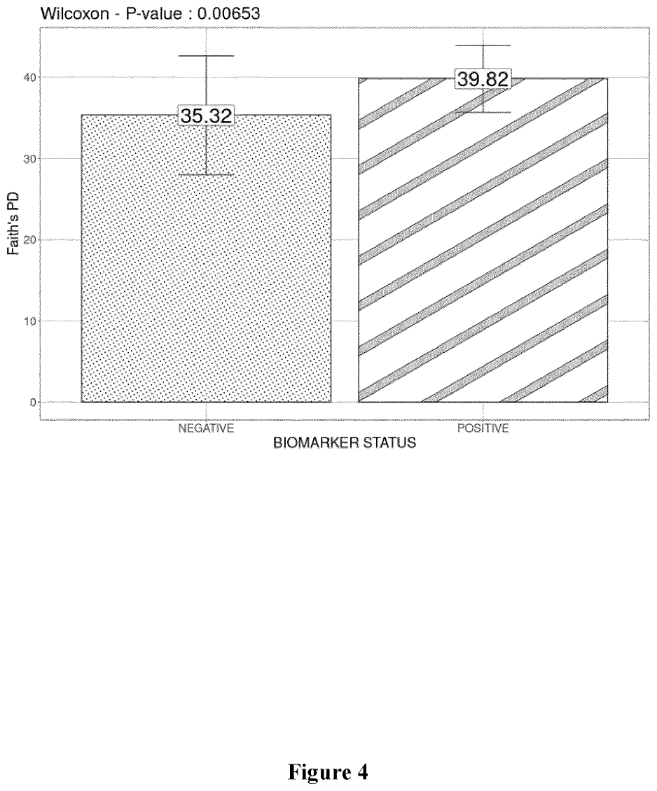

Blastocystis biomarker positive animals were found to exhibit significantly greater «-diversity (Faith's PD) than biomarker negative animals. A Wilcoxon test was applied and resulted in a p-value of 0.00653393 (FIG. 4). Interestingly, this difference was still significant when other α-diversity metrics were considered (observed taxa or even the Shannon index). Animals carrying the Blastocystis biomarker were found to have a statistically significantly higher diversity index than animals not carrying it. The experimental conditions to evaluate these different diversity indices were identical to those developed in the article by Even et al. (Theriogenology, 2020, 142:268-275).

b. Bacterial Composition

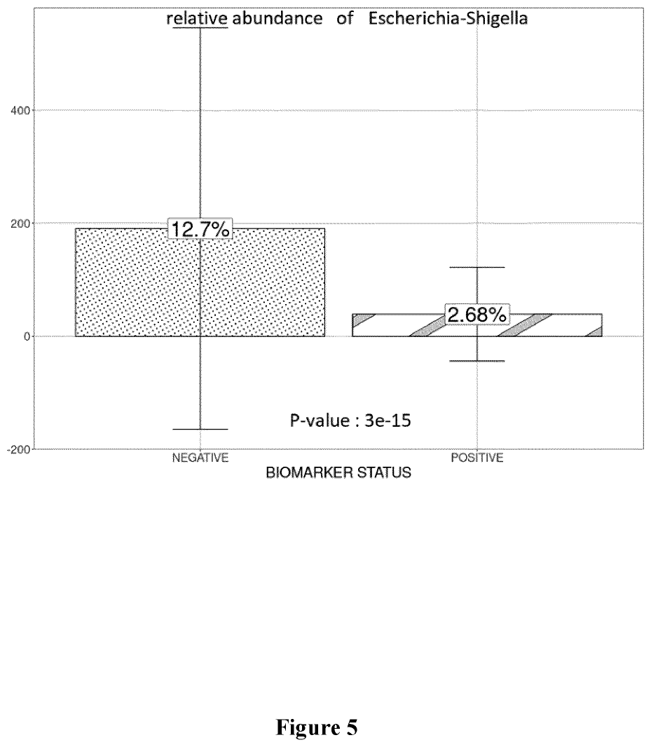

The presence of the biomarker was found to be associated with a drastic decrease in the constitutive genera of the Enterobacteriaceae family. Thus, the genera Shigella and Escherichia were found in significantly decreased relative abundance in the fecal samples of animals carrying the Blastocystis biomarker. Indeed, animals carrying the biomarker were found to show a relative abundance of Shigella-Escherichia genera of 2.68% compared to 12.70% (FIG. 5) for animals negative for the biomarker.

Enterobacteriaceae are a family of bacteria that are hosts of humans and animals in which they reside mainly in the intestine. These Gram-bacteria can be a reservoir of pathogens responsible for subclinical or clinical mastitis, and the relative abundance of this family, as well as the diversity of bacterial genera within it, can increase the risk of acquiring antibiotic resistance genes. Mastitis is one of the three main reasons for culling cows (Young et al., J. Dairy Sci., 1998, 81 (8): 2299-2305). Escherichia coli in some studies has been found to be the most common pathogen isolated from individuals developing grade 3 (severe) mastitis. These grade 3 mastitis cases are systematically treated with antibiotics, and the study (Verbeke et al., Journal of Dairy Science, 2014, 97:6926-6934) concludes that E. coli is isolated in 39% of grade 3 mastitis cases, showing a significant statistical relationship with the severity of the mastitis and the presence of this bacterium (P-value<0.0001).

1-20. (canceled)

21. A method for predicting productive longevity in a dairy cow or for predicting carbon footprint of a dairy cow or for predicting microbiota α-diversity in a dairy cow, the method comprising a step of:
detecting the presence or absence of Blastocystis sp. in a fecal sample obtained from the dairy cow when the dairy cow is a heifer,
wherein the presence of Blastocystis sp. in the fecal sample is indicative of a higher productive longevity in the dairy cow, and the absence of Blastocystis sp. in the fecal sample is indicative of lower productive longevity in the dairy cow;
wherein the presence of Blastocystis sp. in the fecal sample is indicative of a lower carbon footprint for the dairy cow, and the absence of Blastocystis sp. in the fecal sample is indicative of higher carbon footprint for the dairy cow; and
wherein the presence of Blastocystis sp. in the fecal sample is indicative of a higher microbiota α-diversity in the dairy cow, and the absence of Blastocystis sp. in the fecal sample is indicative of lower microbiota α-diversity in the dairy cow.

22. The method according to claim 21, wherein the heifer belongs to a dairy cattle breed.

23. The method according to claim 22, wherein the heifer belongs to a dairy cattle breed selected from the group consisting of Holstein-Friesian, Montbéliarde, Normande, Jersey, Brown Swiss, Guernsey, Ayrshire, and Milking Shorthorn.

24. The method according to claim 21, wherein the heifer is 6 months old or older and is 36 months old or younger.

25. The method according to claim 24, wherein the heifer is between 6 and 20 months old, or between 6 and 18 months old.

26. The method according to claim 21, wherein the step of detecting the presence or absence of Blastocystis sp. in the fecal sample is carried out using a method selected from the group consisting of microscopic examination, in vitro culture, molecular detection, and any combination thereof.

27. The method according to claim 26, wherein the step of detecting the presence or absence of Blastocystis sp. in the fecal sample by molecular detection is carried out using a molecular detection method selected from the group consisting of immunoblots (Western blots), Northern blots, Southern blots, enzyme linked immunosorbent assay (ELISA), immunoprecipitation, immunofluorescence, flow cytometry, immunohistochemistry, nucleic acid hybridization techniques, nucleic acid reverse transcription methods, nucleic acid amplification methods, nucleic acid sequencing methods, and any combination thereof.

28. The method according to claim 27, wherein the step of detecting the presence or absence of Blastocystis sp. in the fecal sample is carried out by polymerase chain reaction (PCR).

29. The method according to claim 27, wherein the step of detecting the presence or absence of Blastocystis sp. in the fecal sample is carried out by real-time quantitative polymerase chain reaction (qPCR).

30. The method according to claim 29, wherein detecting the presence or absence of Blastocystis sp. in the fecal sample by qPCR comprises steps of:
(a) performing an amplifying step comprising contacting the fecal sample, or a nucleic acid isolated therefrom, with a set of primers to produce an amplification product if a nucleic acid from Blastocystis sp. is present in the sample;
(b) performing a hybridizing step comprising contacting the amplification product with a detectable probe; and
(c) detecting the presence of the amplification product, wherein the presence of the amplification product is indicative of the presence of Blastocystis sp. in the fecal sample, and the absence of the amplification product is indicative of the absence of Blastocystis sp. in the fecal sample,
wherein the set of primers to produce an amplification product from Blastocystis sp. comprises forward primers having the sequences set forth in SEQ ID NO: 1 (5′-TTTACTGTGAGAAAATTAGAGTGTTCAAAGC-3′) and SEQ ID NO: 2 (5′-TTTACTGTGAGAAAATTAGAGTGTTTAAAGC-3′) and reverse primers having the sequences set forth in SEQ ID NO: 3 (5′-TGCTTTCGCACTTGTTCATC-3′), SEQ ID NO: 4 (5′-TGCTTTCGCACTAGTTCATC-3′) and SEQ ID NO: 5 (5′-TGCTTTCGCACCTGTTCATC-3′), and wherein the detectable probe has the sequence set for in SEQ ID NO: 6 (5′-CAGTTGGGGGTATTCATATTCA-3′) and is labelled at the 5′-end with a donor fluorescent moiety and at the 3′-end with a quencher.

31. The method according to claim 30, wherein the forward primers having the sequences set forth in SEQ ID NO: 1 and SEQ ID NO: 2; the reverse primers having the sequences set forth in SEQ ID NO: 3, SEQ ID NO: 4 and SEQ ID NO: 5, and the detectable probe having the sequence set for in SEQ ID NO: 6 are comprised in a kit.

32. The method according to claim 30, wherein the detectable probe of SEQ ID NO: 6 comprises at least one locked nucleotide analog at positions: 13, 14, 17, 19 and/or 20 in SEQ ID NO: 6.

33. The method according to claim 32, wherein the detectable probe of SEQ ID NO: 6 comprises one or more locked thymine analogs at positions: 13, 14, 17, 19 and/or 20 in SEQ ID NO: 6.

34. The method according to claim 30, wherein the detectable probe of SEQ ID NO: 6 comprises a locked thymine analog at each of positions 13, 14, 17, 19 and 20 in SEQ ID NO: 6.

35. The method according to claim 34, wherein the detectable probe of SEQ ID NO: 6 comprises a LNA monomer with a thymine base at each of positions 13, 14, 17, 19 and 20 in SEQ ID NO: 6.

36. The method according to claim 30, wherein the detectable probe is labelled at the 5′-end with the donor fluorescent moiety, FAM, and at the 3′-end with the quencher Iowa Black® FQ.

37. The method according to claim 30, further comprising a step of detecting by qPCR an exogenous internal positive control, the bacteriophage lambda, using a forward primer having the sequence set forth in SEQ ID NO: 7 (5′-GGACGTATCATGCTGGCCAA-3′), a reverse primer having the sequence set forth in SEQ ID NO: 8 (5′-GGAAATAGCCTCCGGCTCA-3′), and a detectable probe having the sequence set forth in SEQ ID NO: 9 (5′-TCCTTCGTGATATCGGACGTTGGCTG-3′) and labelled at the 5′-end with the donor fluorescent moiety, HEX, and at the 3′-end with the quencher Iowa Black® FQ and further labelled with the additional quencher ZEN positioned 6 to 15 bases from the 5′ donor fluorescent moiety, in particular 9 bases from the 5′ donor fluorescent moiety.

38. The method according to claim 37, wherein: the forward primers having the sequences set forth in SEQ ID NO: 1 and SEQ ID NO: 2; the reverse primers having the sequences set forth in SEQ ID NO: 3, SEQ ID NO: 4 and SEQ ID NO: 5, and the detectable probe having the sequence set for in SEQ ID NO: 6; the forward primer having the sequence set for in SEQ ID NO: 7; the reverse primer having the sequence set for in SEQ ID NO: 8; and the probe having the sequence set for in SEQ ID NO: 9 as defined in claim 37 are comprised in a kit.

39. A method for improving management of a dairy herd, the method comprising steps of:
(1) predicting productive longevity in a dairy cow at the heifer stage using a method according to claim 21; and
(2) if, in step (1), the heifer is predicted to have a higher productive longevity, selecting the heifer as a replacement heifer for the dairy herd; and
(3) optionally performing artificial insemination of the heifer when the heifer is ready for breeding; or
(4) if, in step (1), the heifer is predicted to have a lower productive longevity, culling the heifer from the dairy herd.

40. The method according to claim 39, wherein improving management of a dairy herd comprises, or results in, at least one of:
optimizing dairy herd renewal;
mitigating greenhouse gas emissions by the dairy herd and decreasing its carbon footprint;
decreasing veterinary costs for the dairy herd; and
improving profitability of the dairy herd.
